(19) United States
(12) Patent Application Publication
(10) Pub. No.: US 2023/0320949 A1
(43) Pub. Date: Oct. 12, 2023

(54) Compositions Having Capsules

(71) Applicant: The Procter & Gamble Company, Cincinnati, OH (US)

(72) Inventors: Mariana B T CARDOSO, Brussels (BE); Andre Martim BARROS, Saint Etienne (BE); Pierre Daniel VERSTRAETE, Saint Lambert (BE); Eric Scott JOHNSON, Hamilton, OH (US); Karl Shiqing WEI, Mason, OH (US)

(21) Appl. No.: 18/127,097

(22) Filed: Mar. 28, 2023

Related U.S. Application Data
(60) Provisional application No. 63/329,899, filed on Apr. 12, 2022.

(51) Int. Cl. A61K 8/11; A61Q 5/02

(52) U.S. Cl. CPC A61K 8/11; A61Q 5/02; A61K 2800/412; A61K 2800/651; A61K 2800/56

(57) ABSTRACT

A composition having one or more leak resistant capsules.

### FIELD OF THE INVENTION

The disclosure relates to compositions comprising capsules for the triggered release of benefit agents.

### BACKGROUND OF THE INVENTION

Microencapsulation is a process where droplets of liquids, particles of solids or gasses are enclosed inside a solid shell and are generally in the micro-size range. The core material is then mechanically separated from the surrounding environment (Jyothi et al., Journal of Microencapsulation, 2010, 27, 187-197). Microencapsulation technology is attracting attention from various fields of science and has a wide range of commercial applications for different industries. Overall, capsules are capable of one or more of (i) providing stability of a formulation or material via the mechanical separation of incompatible components, (ii) protecting the core material from the surrounding environment, (iii) masking or hiding an undesirable attribute of an active ingredient and (iv) controlling or triggering the release of the active ingredient to a specific time or location. All of these attributes can lead to an increase of the shelf-life of several products and a stabilization of the active ingredient in liquid formulations.

Encapsulation can be found in areas such as pharmaceuticals, personal care, textiles, food, coatings and agriculture. In addition, the main challenge faced by microencapsulation technologies in real-world commercial applications is that a complete retention of the encapsulated active within the capsule is required throughout the whole supply chain, until a controlled or triggered release of the core material is applied (Thompson et al., Journal of Colloid and Interface Science, 2015, 447, 217-228). There are significantly limited microencapsulation technologies that are safe for both the environment and human health with a long-term retention and active protection capability that can fulfill the needs of the industry nowadays, especially when it comes to encapsulation of small molecules.

Over the past several years, consumer goods manufacturers have used core-shell encapsulation techniques to preserve actives, such as benefit agents, in harsh environments and to release them at the desired time, which may be during or after use of the consumer goods. Among the several mechanisms that can be used for release of benefit agent, the one commonly relied upon is mechanical rupture of the capsule shell. Selection of mechanical rupture as the release mechanism constitutes another challenge to the manufacturer, as rupture must occur at specific desired times, even if the capsules are subject to mechanical stress prior to the desired release time.

Industrial interest for encapsulation technology has led to the development of several polymeric capsules chemistries which attempt to meet the requirements of low shell permeability, high deposition, targeted mechanical properties and rupture profile. Increased environmental concerns have put the polymeric capsules under scrutiny, therefore manufacturers have started investigating sustainable solutions for the encapsulation of benefit agents. There is ample literature on sustainable capsules based on metal oxide or semi-metal oxides, mainly silica capsules; however, none of the capsules described in the literature provides the right balance of low shell permeability, mechanical properties, deposition, and rupture profile.

Capsules made with silane monomers only are known in the art. Multiple patent applications and academic publications disclose the use of monomers such as tetramethoxysilane (TMOS) and tetraethoxysilane (TEOS). The advantage of using such monomers is that they react faster than prepolymers made from similar monomers, and as such have been the favored option for years. This fast reaction time is due to their higher water solubility once partially hydrolyzed compared to larger precursors, due to the fact that the former have lower molecular weights, which accelerate further the overall hydrolysis kinetics as they are in an excess of water once dispersed in said phase. However, these types of disclosures often use cationic surfactants such as cetyltrimethylammonium chloride (CTAC) or cetyltrimethylammonium bromide (CTAB), supposedly to drive the negatively charged hydrolyzed intermediate reaction species that are dispersed in the water phase towards the oil/water interface.

Without wishing to be bound by theory, what is often the case is that the partially hydrolyzed monomers that are in an excess of water start condensing and forming ever larger particulate sols that are drawn to oil/water interfaces. Ultimately, the system desires to reduce surface energies of dispersed particulate sols by virtue of thermodynamic laws, which favors having the sols at the oil/water interfaces, especially when they grow large. The formation of such particulate sols can eventually lead to a shell around oil droplets and in some cases even shells that are strong enough towards mechanical self-integrity. However, by virtue of the geometrical properties (size, fractal dimensions, shapes etc.) of particulate sols, they are not able to form shells with a dense non-porous network that would provide low shell permeability.

In addition, WO 2011/131644 discloses capsules with a semi-metal organic shell by joining together nanoparticles with the use of an oil soluble semi-metal precursor. However, the reference does not disclose a second shell component. In the present invention it has been found that a selective choice of nanoparticles and precursors coupled with a second shell component provides capsules that have reduced permeability and increased mechanical integrity.

Without wishing to be bound by theory, Applicant has surprisingly found that a careful selection of primary shell components, secondary shell components, nanoparticles, core-shell ratio, and thickness of the shell allows production of metal oxide or semi-metal oxide based capsules, which in combination with certain compositions provide those compositions with improved performance properties.

### SUMMARY OF THE INVENTION

A haircare composition is provided that comprises a surfactant; at least one of a fatty alcohol, cationic polymer, or a mixture thereof; one or more capsules; a capsule comprising a core and a shell surrounding the core; wherein the core comprises perfume raw materials; wherein the shell comprises-a substantially inorganic first shell component comprising a condensed layer and a nanoparticle layer; wherein the condensed layer comprises a condensation product of a precursor; wherein the nanoparticle layer comprises inorganic nanoparticles; and wherein the condensed layer is disposed between the core and the nanoparticle layer; an inorganic second shell component surrounding the first shell component, wherein the second shell component surrounds the nanoparticle layer;
wherein the precursor comprises at least one compound of Formula (I), Formula (II), or mixture thereof,
   wherein Formula (I) is (MvOzYn)w,
   wherein Formula (II) is (MvOzYnR1p)w,
wherein for Formula (I), Formula (II), or the mixture thereof:
   each M is independently selected from the group consisting of silicon, titanium, and aluminum,
   v is the valence number of M and is 3 or 4,
   z is from 0.5 to 1.6,
   each Y is independently selected from —OH, —OR², halogen,

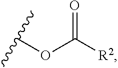

NH₂, —NHR², —N(R²)₂, and

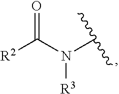

wherein R2 is a C1 to C20 alkyl, C1 to C20 alkylene, C6 to C22 aryl, or a 5-12 membered heteroaryl, wherein the heteroaryl comprises from 1 to 3 ring heteroatoms selected from O, N, and S, wherein R3 is a H, C1 to C20 alkyl, C1 to C20 alkylene, C6 to C22 aryl, or a 5-12 membered heteroaryl, wherein the heteroaryl comprises from 1 to 3 ring heteroatoms selected from O, N, and S,
w is from 2 to 2000;
wherein for Formula (I),
   n is from 0.7 to (v-1); and
wherein for Formula (II),
   n is from 0 to (v-1);
   each R1 is independently selected from the group consisting of: a C1 to C30 alkyl; a C1 to C30 alkylene; a C1 to C30 alkyl substituted with a member selected from the group consisting of a halogen, —OCF3, —NO2, —CN, —NC, —OH, —OCN, —NCO, alkoxy, epoxy, amino, mercapto, acryloyl, —CO2H, —C(O)-alkyl, —C(O)O-aryl, and —C(O)O-heteroaryl; and a C1 to C30 alkylene substituted with a member selected from the group consisting of a halogen, —OCF3, —NO2, —CN, —NC, —OH, —OCN, —NCO, alkoxy, epoxy, amino, mercapto, acryloyl, —C(O)OH, —C(O)O-alkyl, —C(O)O-aryl, and —C(O)O-heteroaryl; and
   p is a number that is greater than zero and is up to pmax,
      wherein pmax=60/[9*Mw(R1)+8],
wherein Mw(R1) is the molecular weight of the R1 group.

A personal care composition is provided that comprises a surfactant; skin conditioning agent, and one or more capsules; a capsule comprising a core and a shell surrounding the core; wherein the core comprises perfume raw materials; wherein the shell comprises—a substantially inorganic first shell component comprising a condensed layer and a nanoparticle layer; wherein the condensed layer comprises a condensation product of a precursor; wherein the nanoparticle layer comprises inorganic nanoparticles; and wherein the condensed layer is disposed between the core and the nanoparticle layer; an inorganic second shell component surrounding the first shell component, wherein the second shell component surrounds the nanoparticle layer;
wherein the precursor comprises at least one compound of Formula (I), Formula (II), or mixture thereof,
   wherein Formula (I) is (MvOzYn)w,
   wherein Formula (II) is (MvOzYnR1p)w,
wherein for Formula (I), Formula (II), or the mixture thereof:
   each M is independently selected from the group consisting of silicon, titanium, and aluminum,
   v is the valence number of M and is 3 or 4,
   z is from 0.5 to 1.6,
   each Y is independently selected from —OH, —OR², halogen,

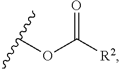

—NH₂, —NHR², —N(R²)₂, and

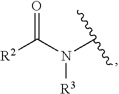

wherein R2 is a C1 to C20 alkyl, C1 to C20 alkylene, C6 to C22 aryl, or a 5-12 membered heteroaryl, wherein the heteroaryl comprises from 1 to 3 ring heteroatoms selected from O, N, and S, wherein R3 is a H, C1 to C20 alkyl, C1 to C20 alkylene, C6 to C22 aryl, or a 5-12 membered heteroaryl, wherein the heteroaryl comprises from 1 to 3 ring heteroatoms selected from O, N, and S,
w is from 2 to 2000;
wherein for Formula (I),
   n is from 0.7 to (v-1); and
wherein for Formula (II),
   n is from 0 to (v-1);
   each R1 is independently selected from the group consisting of: a C1 to C30 alkyl; a C1 to C30 alkylene; a C1 to C30 alkyl substituted with a member selected from the group consisting of a halogen, —OCF3, —NO2, —CN, —NC, —OH, —OCN, —NCO, alkoxy, epoxy, amino, mercapto, acryloyl, —CO2H, —C(O)-alkyl, —C(O)O-aryl, and —C(O)O-heteroaryl; and a C1 to C30 alkylene substituted with a member selected from the group consisting of a halogen, —OCF3, —NO2, —CN, —NC, —OH, —OCN, —NCO, alkoxy, epoxy, amino, mercapto, acryloyl, —C(O)OH, —C(O)O-alkyl, —C(O)O-aryl, and —C(O)O-heteroaryl; and
   p is a number that is greater than zero and is up to pmax,
      wherein pmax=60/[9*Mw(R1)+8],
wherein Mw(R1) is the molecular weight of the R1 group.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 shows a schematic illustration of the method of making capsules with a first shell component, prepared with a hydrophobic core.

FIG. 2 shows a schematic illustration of a capsule with a first shell component and a second shell component.

FIG. 3 is a scanning electron microscopy image of a capsule.

FIG. 4 is a graph of leakage results of capsules of this invention and comparative capsules in hair care composition 1, as detailed in TABLE 7.

FIG. 5 is a graph of leakage results of capsules of this invention and comparative capsules in hair care composition 2, as detailed in TABLE 7.

FIG. 6 is a graph of leakage results of capsules of this invention and comparative capsules in hair care composition 3, as detailed in TABLE 8

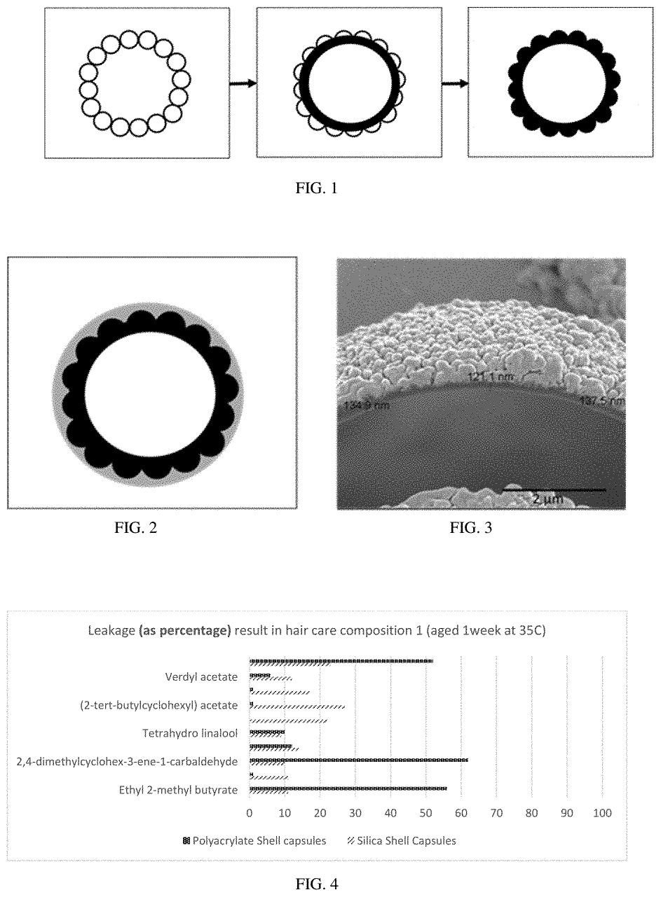

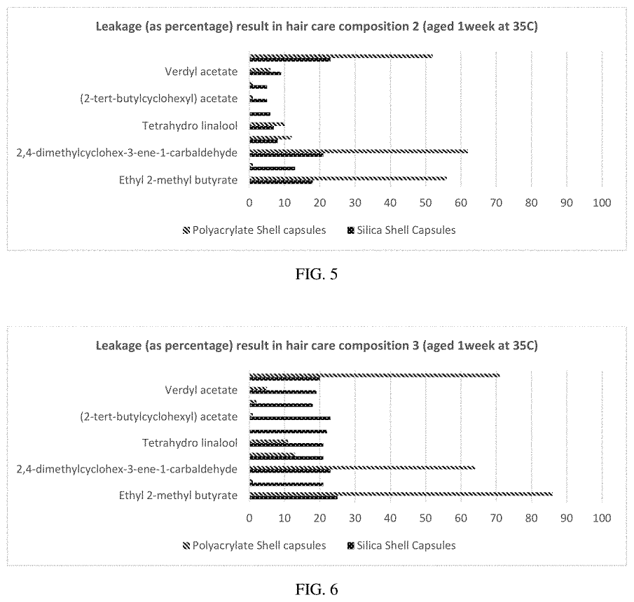

### DETAILED DESCRIPTION OF THE INVENTION

The present disclosure relates to liquid hair care, personal care and shave care compositions comprising populations of capsules having perfume raw materials. The shells of the capsules contain inorganic materials, the selection of which results in improved mechanical properties and low and/or consistent permeability.

It has been found that the capsules present in the disclosed inventive compositions work surprisingly well in controlling leakage of perfume raw materials, resulting in relatively low and consistent perfume leakage. Without wishing to be bound by theory, it is believed that the leakage of perfume raw materials is driven by radically different mechanisms for shells containing highly crosslinked inorganic materials compared to shells containing organic polymeric materials. Specifically, the diffusion of small molecules such as perfume raw materials (“PRMs”) across a homogenous organic polymeric shell is similar to the diffusion mechanism across a homogeneous polymeric membrane. In this case, the permeability of the polymeric membrane for a given solute depends both on the polymer free volume (impacted by degree of crystallinity and cross-linked density) as well as the relative solubility of the solute for the polymer. Since different PRMs will have different ranges of relevant physical and chemical properties (e.g., molecular weight and polarity), the rates of diffusion are not uniform for a given set of PRMs when the physical and chemical properties are also not uniform.

On the other hand, it is believed that diffusion of small molecules across a highly crosslinked inorganic shell occurs primarily through the microchannels formed by the percolating network of micropores present in the shell. Such highly crosslinked inorganic shell can be obtained by using a second shell component in combination with a first shell component, as disclosed with the present disclosure. In this case, it is believed that the permeability of the inorganic shell primarily depends on the number, density, and dimensions of the microchannels that are effectively connecting the core and continuous phases, which can result in the PRM leakage rates being relatively uniform or consistent with respect to each other, as well as being relatively low. As the various PRMs leak from the disclosed capsules in the disclosed compositions at relatively consistent rates, it is further believed that the intended character of the perfume is maintained, leading to a more satisfactory and consistent olfactory performance during usage and storage of the present hair care, personal care and shave care compositions.

### Skin Conditioning Agent

The compositions of this invention may comprise one or more skin conditioning agents.

The one or more skin conditioning agents may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the silicones can be in an emulsified form with droplet size of about 10 nm to about 30 micrometers Other solid or semi-solid conditioning agents may be present in the composition including high melting temperature fatty alcohols, acids, esters, amides or oligomers from unsaturated esters, alcohols, amides. The oligomeric esters may be the result of oligomerization of naturally-occurring unsaturated glyceride esters. Such solid or semi-solid conditioning agents may be added or present as mixtures with organic oils.

The one or more skin conditioning agent may also comprise at least one organic conditioning material such as oil or wax, either alone or in combination with other conditioning agents, such as the silicone conditioning agents described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

The one or more skin conditioning agent may further comprise a conditioning agent that is at least one of humectants or moisturizers, each can be present at a level of from about 0.01% to about 40%, more preferably from about 0.1% to about 30%, and even more preferably from about 0.5% to about 15% by weight of the composition. These materials include, but are not limited to, guanidine; urea; glycolic acid and glycolate salts (e.g. ammonium and quaternary alkyl ammonium); lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; sucrose polyester; petrolatum; and mixtures thereof.

Suitable moisturizers, also referred to in the present invention as humectants, include urea, guanidine, glycolic acid and glycolate salts (e.g. ammonium and quaternary alkyl ammonium), lactic acid and lactate salts (e.g. ammonium and quaternary alkyl ammonium), aloe vera in any of its variety of forms (e.g. aloe vera gel), polyhydroxy alcohols (such as sorbitol, glycerol, hexanetriol, propylene glycol, hexylene glycol and the like), polyethylene glycol, sugars and starches, sugar and starch derivatives (e.g. alkoxylated glucose), hyaluronic acid, lactamide monoethanolamine, acetamide monoethanolamine, and mixtures thereof.

The one or more skin conditioning agent may also comprise a benefit agent, which can be a liquid benefit agent. A liquid benefit agent is considered liquid if that is its natural state at room temperature (i.e. 23° C.). A liquid benefit agent can have a viscosity of less than about 1000 cP, less than about 800 cP, or less than about 600 cP, and can be measured with a standard rheometer.

The liquid benefit agent can have a hydrophobic component. The hydrophobic component can be, for example, a water-dispersible, non-volatile liquid. The water-dispersible, non-volatile liquid benefit agents can have a Vaughn Solubility Parameter (VSP) ranging from about 5 to about 14. Non-limiting examples of hydrophobic benefit materials having VSP values ranging from about 5 to about 14 include the following: Cyclomethicone (5.9), Squalene (6.0), Isopropyl Palmitate (7.8), Isopropyl Myristate (8.0), Castor Oil (8.9), Cholesterol (9.6), Butylene Glycol (13.2), soy bean oil, olive oil (7.87), mineral oil (7.1), and combinations thereof.

Non-limiting examples of glycerides suitable for use as liquid benefit agents herein can include castor oil, safflower oil, corn oil, walnut oil, peanut oil, olive oil, cod liver oil, almond oil, avocado oil, palm oil, sesame oil, soybean oil, vegetable oils, sunflower seed oil, coconut oil, cottonseed oil, jojoba oil, and combinations thereof.

Non-limiting examples of glyceride derivatives suitable for use as liquid benefit agents herein can include cationic derivatives, amino acid derivatives, alkanolamide derivatives, esterified derivatives, ether derivatives, hydrogenated derivatives, and combinations thereof.

Non-limiting examples of metathesized oligomers suitable for use as liquid benefit agents herein can include oligomers derived from metathesis of unsaturated polyol esters, for example. Exemplary metathesized unsaturated polyol esters and their starting materials are set forth in U.S. Patent Application U.S. 2009/0220443 A1, which is incorporated herein by reference. The unsaturated polyol ester is an unsaturated ester of glycerol. Sources of unsaturated polyol esters of glycerol include synthesized oil, plant oils, algae oils, bacterial derived oils, and animal oils, combinations of theses, and the like. Representative examples of plant oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, tung oil, castor oil, high erucic rape oils, Jatropha oil, combinations of theses, and the like. Representative examples of animal oils include fish oil and the like. A representative example of a synthesized oil includes tall oil, which is a byproduct of wood pulp manufacture.

Other examples of unsaturated polyol esters include diesters such as those derived from ethylene glycol or propylene glycol, esters such as those derived from pentaerythritol or dipentaerythritol, or sugar esters such as SEFOSE®. Non-limiting examples of sucrose polyesters suitable for use include SEFOSE® 1618S, SEFOSE® 1618U, SEFOSE® 1618S B6, SEFOSE® 1618U B6, Sefa Cottonate, Sefa C895, Sefa C1095, SEFOSE® 1618S B4.5, all available from The Procter and Gamble Co. of Cincinnati, Ohio. Other examples of suitable natural polyol esters may include but not be limited to sorbitol esters, maltitol esters, sorbitan esters, maltodextrin derived esters, xylitol esters, and other sugar derived esters. The poloyl ester oligomers may also be modified further by partial hydroformylation of the unsaturated functionality to provide one or more OH groups and an increase in the oligomer hydrophilicity.

Non-limiting examples of hydrocarbons suitable for use as liquid benefit agents herein can include carbon chain length of about C6 or higher including alkanes, polyalkanes, olefins, polyolefins and combinations thereof. Non-limiting examples include mineral oil.

Non-limiting examples of glyceride derivatives for use as liquid benefit agents here in can include cationic derivatives, amino acid derivatives, alkanolamide derivatives, esterified derivatives, ether derivatives, hydrogenated or partially hydrogenated oils and their derivatives, and combination thereof.

Non-limiting examples of alkyl esters suitable for use as liquid benefit agents herein can include isopropyl esters of fatty acids and long chain esters of long chain (i.e. C10-C16) fatty acids, non-limiting examples of which can include isopropyl palmitate, isohexyl palmitate and isopropyl myristate.

Non-limiting examples of silicone oils suitable for use as hydrophobic liquid skin benefit agents herein can include dimethicone copolyol, dimethylpolysiloxane, diethylpolysiloxane, mixed C1-C30 alkyl polysiloxanes, phenyl dimethicone, dimethiconol, and combinations thereof. Nonlimiting examples of silicone oils useful herein are described in U.S. Pat. No. 5,011,681. Still other suitable hydrophobic skin benefit agents can include milk triglycerides (e.g., hydroxylated milk glyceride) and polyol fatty acid polyesters.

The benefit agent may also be non-liquid. Some examples of non-liquid benefit agents include hydrocarbons. Non-limiting examples of hydrocarbons suitable for use as non-liquid benefit agents herein can include petrolatum, microcrystalline wax, polyalkanes, polyolefins, and combinations thereof.

Non-limiting examples of glycerides suitable for use as non-liquid benefit agents herein can include plant waxes, animal fats, hydrogenated or partially hydrogenated plant oils, e.g. shea butter, hydrogenated soybean oil, hydrogenated palm, lanolin, lard, and combinations thereof.

Non-limiting examples of metathesized glycerides suitable for use as non-liquid benefit agents herein can include metathesized palm oil, hydrogenated or partially hydrogenated metathesized soybean oil and canola oil, and combinations thereof.

Non-limiting examples of alkyl esters suitable for use as non-liquid benefit agents herein can include isopropyl esters of fatty acids and long chain esters of long chain (i.e. C10-C24) fatty acids, e.g., cetyl ricinoleate, non-limiting examples of which can include cetyl riconoleate and stearyl riconoleate. Other examples can include hexyl laurate, isohexyl laurate, myristyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as non-liquid benefit agents can include oleyl myristate, oleyl stearate, oleyl oleate, and combinations thereof.

Non-limiting examples of polyglycerin fatty acid esters suitable for use as non-liquid benefit agents herein can include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as non-liquid benefit agents herein can include lanolin, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Non-limiting examples of silicones suitable for use hydrophobic liquid skin benefit agents can include silicone elastomers.

Other suitable benefit agents are described in U.S. Patent Application Publication No. 2012/0009285.

The benefit phase may also comprise a crystalline hydrophobic ethylene copolymer. The ethylene copolymers are random copolymers and may be present from about 0.01% to about 5% by weight of the personal care composition. The crystalline hydrophobic ethylene copolymer may be present from about 0.05% to about 2% by weight of the personal care composition. As another example, the crystalline hydrophobic ethylene copolymer may be present from about 0.1% to about 1.5% by weight of the personal care composition.

The crystalline hydrophobic ethylene copolymer contains at least 40% ethylene monomer by weight of the crystalline hydrophobic ethylene acrylate copolymer. The crystalline hydrophobic ethylene copolymer can contain from about 40%, 50%, 60%, 70%, 80%, 90%, 95%, 96%, 97%, to about 99%, 98%, 97%, 96%, 95%, 90%, 80%, 70%, 60%, 50%, or any combination thereof to form a range, of ethylene monomer.

In addition, the crystalline hydrophobic ethylene copolymer can comprise an acrylate monomer. The polymer may contain about 1% to about 60%, by weight of the polymer, of an acrylate monomer. The acrylate monomer may be defined by the following formula: (R¹)(R²)C═C(R³)(COOR⁴), wherein, each R¹, R², and R³ is independently H or C₁-C₄-alkyl, in one example H or methyl, in another example two of R¹, R², and R³ are H and the other is H or methyl, in another example R¹, R², and R³ are all H; and R4 is C₁-C₂₀-alkyl, or is selected from straight-chain and branched alkyl groups having from 4 to 20, from 6 to 20, from 8 to 20, or from 9 to 20 carbon atoms.

Some examples of suitable crystalline hydrophobic ethylene acrylate copolymers include ethylene:propylheptylacrylate, ethylene:propylheptylacrylate:vinyl acetate, and combinations thereof. A suitable crystalline hydrophobic ethylene acrylate copolymer can include 86.2% ethylene:13.8% propylheptylacrylate; 90.4% ethylene:9.6% propylheptylacrylate; 96% ethylene:4% propylheptylacrylate; or 81.8% ethylene:9.6% propylheptylacrylate: 8.6% vinyl acetate.

The crystalline hydrophobic ethylene copolymer can comprise a vinyl actetate monomer. The vinyl acetate monomer may be defined by the following formula: (R¹⁰)(R¹¹)C═C(R⁹)(COR¹²), wherein R⁹ is independently H or C₁-C₄-alkyl, one of R¹⁰ and R¹¹ is —C(O)R¹³ and the other is H or C₁-C₄-alkyl; and R² and R³ are each independently —OH or C₁-C₂₀-alkoxy; or R¹² and R¹³ together from an —O— group.

In addition, a crystalline hydrophobic ethylene acrylate copolymer can include a combination of ethylene, propylheptylacrylate, and an additional monomer. This additional monomer can be up to 10%, by weight of the copolymer. This additional monomer can be represented as (R⁵)(R⁶)C═C(R⁷)(OCOR⁸) wherein, each R⁵, R⁶, and R⁷ is independently H or C₁-C₄-alkyl, preferably H or methyl, more preferable two of R⁵, R⁶, and R⁷ are H and the other is H or methyl, in particular R⁵, R⁶, and R⁷ are all H; and R⁸ is C₁-C₂₀-alkyl, preferably C₁-C₉-alkyl, more preferably C₁-C₃-alkyl, specifically either or methyl, and especially methyl. A suitable example of this additional monomer is vinyl acetate.

### Hair Care Composition

The capsules of the current invention can be used in hair care compositions to provide one or more benefits, including freshness, malodor removal, softness and styling. The hair care compositions of the present invention can be in different forms. Non-limiting examples of said forms are: shampoos, conditioning shampoos, pet shampoo, leave-on treatments, sprays, liquids, pastes, Newtonian or non-Newtonian fluids, gels, and sols.

The hair care composition may comprise capsules having at least one benefit agent at a level where upon directed use, promotes one or more benefits without detriment to the hair. Such benefit agent may comprise a perfume, an essential oil, a silicone, a wax and mixtures thereof. The perfume may comprise a single perfume raw material or a mixture of perfume raw materials. Examples of essential oils are argan oil, lavender oil, peppermint oil, rosemary oil, thyme oil, cedarwood oil, lemongrass oil, ylang-ylang oil and mixtures thereof.

In embodiments of the present invention, said hair care composition comprises between about 0.01 wt % to about 15 wt % of at least one benefit agent encapsulated in a capsule. In another embodiment, said hair care composition comprises between about 0.05 wt % to about 8 wt % of at least one benefit agent encapsulated. In another embodiment, said hair care composition comprises between about 0.1 wt % to about 5 wt % of at least one benefit agent encapsulated.

In addition to capsules, the hair care compositions of the present invention may also include detersive surfactants, aqueous carriers, shampoo gel matrixes, and other additional ingredients.

### Detersive Surfactant

Hair care compositions may comprise one or more detersive surfactants, which provide cleaning performance to the composition. The one or more detersive surfactants in turn may comprise an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety.

The concentration of the detersive surfactant component in the hair care composition should be sufficient to provide the desired cleaning and lather performance, and generally ranges from 2 wt % to about 50 wt %, from about 5 wt % to about 30 wt %, from about 8 wt % to about 25 wt %, from about 10 wt % to about 20 wt %, about 5 wt %, about 10 wt %, about 12 wt %, about 15 wt %, about 17 wt %, about 18 wt %, or about 20 wt %.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the hair care composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment, the anionic surfactant is sodium lauryl sulfate or sodium laureth sulfate.

Suitable amphoteric or zwitterionic surfactants for use in the hair care composition herein include those which are known for use in shampoo or other personal care cleansing. Concentrations of such amphoteric surfactants range from about 0.5 wt % to about 20 wt %, and from about 1 wt % to about 10 wt %. Non limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the hair care composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present hair care composition include cocoamphoacetate, cocoamphodiacetate, lauroamphoacetate, lauroamphodiacetate, and mixtures thereof.

Zwitterionic detersive surfactants suitable for use in the hair care composition include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In another embodiment, zwitterionics such as betaines are selected.

Non limiting examples of other anionic, zwitterionic, amphoteric or optional additional surfactants suitable for use in the hair care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

The hair care composition may also comprise a shampoo gel matrix, an aqueous carrier, and other additional ingredients described herein.

### Aqueous Carrier

Hair care compositions may comprise a first aqueous carrier. The level and species of the carrier are selected according to the compatibility with other components and other desired characteristic of the product. Accordingly, the formulations of the hair care composition can be in the form of pourable liquids (under ambient conditions). Such compositions will therefore typically comprise a first aqueous carrier, which is present at a level of at least 20 wt %, from about 20 wt % to about 95 wt %, or from about 60 wt % to about 85 wt %. The first aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The first aqueous carriers useful in the hair care composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

In embodiments of the present invention, the aqueous carrier is substantially water. In a further embodiment, deionized water may be used. Water from natural sources including mineral cations can also be used, depending on the desired characteristic of the product. Generally, the compositions of the present invention comprise from about 0% to about 99%, in an embodiment from about 50% to about 95%, in a further embodiment from about 70% to about 90%, and in a further embodiment from about 80% to about 90% water.

### Shampoo Gel Matrix

In embodiments, hair care compositions described herein may comprise a shampoo gel matrix. The shampoo gel matrix comprises (i) from about 0.1% to about 20% of one or more fatty alcohols, alternative from about 0.5% to about 14%, alternatively from about 1% to about 10%, alternatively from about 6% to about 8%, by weight of the shampoo gel matrix; (ii) from about 0.1% to about 10% of one or more shampoo gel matrix surfactants, by weight of the shampoo gel matrix; and (iii) from about 20% to about 95% of an aqueous carrier, alternatively from about 60% to about 85% by weight of the shampoo gel matrix.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20 are suitable.

The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carrier useful herein includes water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

### Additional Ingredients

Skin conditioning agent, as described above, in particular these can be the silicone conditioning agents and organic conditioning agents.

Hair care composition of the present invention may also further comprise a nonionic polymer.

According to an embodiment, the conditioning agent for use in the hair care composition of the present invention may include a polyalkylene glycol polymer. For example, polyalkylene glycols having a molecular weight of more than about 1000 are useful herein. Useful are those having the following general formula (VIII):

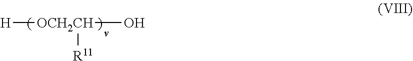

wherein R¹¹ is selected from the group consisting of H, methyl, and mixtures thereof; and v is the number of ethoxy units. The polyalkylene glycols, such as polyethylene glycols, can be included in the hair care compositions of the present invention at a level of from about 0.001 wt. % to about 10 wt. %. In an embodiment, the polyethylene glycol is present in an amount up to about 5 wt. % based on the weight of the composition. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

The hair care compositions of the present invention may further comprise a deposition aid, such as a cationic polymer. Cationic polymers useful herein are those having an average molecular weight of at least about 5,000, alternatively from about 10,000 to about 10 million, and alternatively from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

The cationic polymer can be included in the hair care compositions of the present invention at a level of from about 0.001 wt. % to about 10 wt. %. In one embodiment, the cationic polymer is present in an amount up to about 5 wt % based on the weight of the composition.

In embodiments, the hair care composition further comprises one or more additional benefit agents. The benefit agents comprise a material selected from the group consisting of anti-dandruff agents, anti-fungal agents, anti-itch agents, anti-bacterial agents, anti-microbial agents, moisturization agents, anti-oxidants, vitamins, lipid soluble vitamins, chelants, perfumes, brighteners, enzymes, sensates, attractants, dyes, pigments, bleaches, and mixtures thereof.

Hair care compositions may comprise an anti-dandruff active, which may be an anti-dandruff active particulate. In an embodiment, the anti-dandruff active is selected from the group consisting of: pyridinethione salts; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt.

Pyridinethione particulates are suitable particulate anti-dandruff actives. In an embodiment, the anti-dandruff active is a 1-hydroxy-2-pyridinethione salt and is in particulate form. In an embodiment, the concentration of pyridinethione anti-dandruff particulate ranges from about 0.01 wt. % to about 5 wt. %, or from about 0.1 wt. % to about 3 wt. %, or from about 0.1 wt. % to about 2 wt. %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. Nos. 2,809,971; 3,236,733; 3,753,196; 3,761,418; 4,345,080; 4,323,683; 4,379,753; and 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, charcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

Azole anti-microbials may be an imidazole that is at least one of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the hair care composition, the azole anti-microbial active is included in an amount of from about 0.01 wt. % to about 5 wt. %, or from about 0.1 wt. % to about 3 wt. %, or from about 0.3 wt. % to about 2 wt. %. In an embodiment, the azole anti-microbial active is ketoconazole. In an embodiment, the sole anti-microbial active is ketoconazole.

Embodiments of the hair care composition may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In embodiments, the composition comprises an effective amount of a zinc-containing layered material. In an embodiment, the composition comprises from about 0.001 wt. % to about 10 wt. %, or from about 0.01 wt. % to about 7 wt. %, or from about 0.1 wt. % to about 5 wt. % of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In embodiments the ZLM may be at least one of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which may be synthetic, are layered double hydroxides. In an embodiment, the ZLM is a layered double hydroxide conforming to the formula [M²⁺₁₋ₓM³⁺ₓ(OH)₂]^{x+} A^{m-}_{x/m}·nH₂O wherein some or all of the divalent ions (M²⁺) are zinc ions (Crepaldi, E L, Pava, P C, Tronto, J, Valim, J B J. Colloid Interfac. Sci. 2002,248,429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺ Aⁿ⁻_{(1-3y)/n}·nH₂O where the two metal ions (M²⁺) may be the same or different. If they are the same and represented by zinc, the formula simplifies to [Zn₁₊ₓ(OH)₂]^{2x+} 2x A⁻·nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replaces the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

In embodiments, the rinse-off hair care composition may comprise a rheology modifier. The rheology modifier increases the substantivity and stability of the composition, improves feel and consumer's use experience (e.g. non-dripping, spreadability, etc). Any suitable rheology modifier can be used. In an embodiment, the hair care composition may comprise from about 0.05% to about 10% of a rheology modifier, in a further embodiment, from about 0.1% to about 10% of a rheology modifier, in yet a further embodiment, from about 0.5% to about 2% of a rheology modifier, in a further embodiment, from about 0.7% to about 2% of a rheology modifier, and in a further embodiment from about 1% to about 1.5% of a rheology modifier. In an embodiment, the rheology modifier may be a polyacrylamide thickener. In an embodiment, the rheology modifier may be a polymeric rheology modifier.

In embodiments, the rinse-off hair care composition may comprise rheology modifiers that are homopolymers based on acrylic acid, methacrylic acid or other related derivatives, non-limiting examples include polyacrylate, polymethacrylate, polyethylacrylate, and polyacrylamide.

In embodiments, the rheology modifiers may be alkali swellable and hydrophobically-modified alkali swellable acrylic copolymers or methacrylate copolymers non-limiting examples include acrylic acid/acrylonitrogen copolymer, acrylates/steareth-20 itaconate copolymer, acrylates/ceteth-20 itaconate copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/vinylneodecanoate crosspolymer, and acrylates/C10-C30 alkyl acrylate crosspolymer.

In embodiments, rheology modifiers may be crosslinked acrylic polymers, a non-limiting example includes carbomers.

In embodiments, rheology modifiers may be alginic acid-based materials; non-limiting examples include sodium alginate, and alginic acid propylene glycol esters.

In embodiments, rheology modifiers may be an associative polymeric thickeners, non-limiting examples include: Hydrophobically modified cellulose derivatives; Hydrophobically modified alkoxylated urethane polymers, nonlimiting example include PEG-150/decyl alcohol/SMDI copolymer, PEG-150/stearyl alcohol/SMDI copolymer, polyurethane-39; Hydrophobically modified, alkali swellable emulsions, non-limiting examples include hydrophobically modified polyacrylates, hydrophobically modified polyacrylic acids, and hydrophobically modified polyacrylamides; hydrophobically modified polyethers wherein these materials may have a hydrophobe that can be selected from cetyl, stearyl, oleayl, and combinations thereof, and a hydrophilic portion of repeating ethylene oxide groups with repeat units from 10-300, in another embodiment from 30-200, in a further embodiment from 40-150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

In embodiments, the rheology modifier may be cellulose and derivatives; nonlimiting examples include microcrystalline cellulose, carboxymethylcelluloses, hydroxyethylcellulose, hydroxypropylcellulose, hydroxypropylmethylcellulose, methylcellulose, ethylcellulose, nitro cellulose, cellulose sulfate, cellulose powder, and hydrophobically modified celluloses In embodiments, the rheology modifier may be a guar and guar derivatives; nonlimiting examples include hydroxypropyl guar, and hydroxypropyl guar hydroxypropyl trimonium chloride.

In embodiments, the rheology modifier may be polyethylene oxide, polypropylene oxide, and POE-PPO copolymers.

In embodiments, the rheology modifier may be polyvinylpyrrolidone, crosslinked polyvinylpyrrolidone and derivatives. In a further embodiment, the rheology modifier may be polyvinyalcohol and derivatives.

In embodiments, the rheology modifier may be polyethyleneimine and derivatives.

In embodiments, the rheology modifier may be silicas; nonlimiting examples include fumed silica, precipitated silica, and silicone-surface treated silica.

In embodiments, the rheology modifier may be water-swellable clays non-limiting examples include laponite, bentolite, montmorilonite, smectite, and hectonite.

In embodiments, the rheology modifier may be gums nonlimiting examples include xanthan gum, guar gum, hydroxypropyl guar gum, Arabia gum, tragacanth, galactan, carob gum, karaya gum, and locust bean gum.

In embodiments, the rheology modifier may be, dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran.

In embodiments, the composition of the present invention may comprise suspending agents including crystalline suspending agents which can be categorized as acyl derivatives, long chain amine oxides, and mixtures thereof. These suspending agents are described in U.S. Pat. No. 4,741,855. These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents. Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA). Examples of suitable long chain amine oxides for use as suspending agents include alkyl dimethyl amine oxides, e.g., stearyl dimethyl amine oxide. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

Non-limiting examples of rheology modifiers include acrylamide/ammonium acrylate copolymer (and)polyisobutene (and) polysorbate 20, acrylamide/sodium acryloyldimethyl taurate copolymer/isohexadecane/polysorbate 80, acrylates copolymer; acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil, C12-16 alkyl PEG-2 hydroxypropylhydroxyethyl ethylcellulose (HM-EHEC), carbomer, crosslinked polyvinylpyrrolidone (PVP), dibenzylidene sorbitol, hydroxyethyl ethylcellulose (EHEC), hydroxypropyl methylcellulose (HPMC), hydroxypropyl methylcellulose (HPMC), hydroxypropylcellulose (HPC), methylcellulose (MC), methylhydroxyethyl cellulose (MEHEC), PEG-150/decyl alcohol/SMDI copolymer, PEG-150/stearyl alcohol/SMDI copolymer, polyacrylamide/C13-14 isoparaffin/laureth-7; polyacrylate 13/polyisobutene/polysorbate 20; polyacrylate crosspolymer-6, polyamide-3; polyquaternium-37 (and) hydrogenated polydecene (and) trideceth-6, polyurethane-39, sodium acrylate/acryloyldimethyltaurate/dimethylacrylamide, crosspolymer (and) isohexadecane (and) polysorbate 60; sodium polyacrylate. Exemplary commercially-available rheology modifiers include ACULYN™ 28, Klucel™ M CS, Klucel™ H CS, KluceI™ G CS, SYLVACLEAR™ AF1900V, SYLVACLEAR™ PA1200V, Benecel™ E10M, Benecel™ K35M, Optasense™ RMC70, ACULYN™33, ACULYN™46, ACULYN™22, ACULYN™44, Carbopol Ultrez™ 20, Carbopol Ultrez™ 21, Carbopol Ultrez™ 10, Carbopol Ulterez™ 30, Carbopol™ 1342, Carbopol™ 934, Carbopol™ 940, Carbopol™ 950, Carbopol™ 980, and Carbopol™ 981, Acrysol™ 22, Sepigel™ 305, Simulgel™600, Sepimax Zen, Simulquat HC 305 and combinations thereof.

### Personal Care Composition

The capsules of the present invention can be used in personal care compositions to provide one or more benefits, including freshness and/or softeness. Personal Care Compositions are intended for topical application to the skin, including topical prescription medications, over-the-counter medications, behind-the-counter medications, cosmetics, consumer goods, and combinations thereof. The personal care compositions of the present invention can be in different forms. Non-limiting examples of said forms are: bar soap, body wash, moisturizing body wash, shower gels, skin cleansers, cleansing milks, in shower body moisturizer, shaving preparations, cleansing compositions used in conjunction with a disposable cleansing cloth, sprays, liquids, pastes, Newtonian or non-Newtonian fluids, gels, and sols.

Personal care compositions may comprise capsules having at least one benefit agent at a level where upon directed use, promotes one or more benefits. In embodiments of the present invention, said personal care composition may comprise between about 0.01 wt % to about 15 wt % of at least one benefit agent encapsulated in said capsules. In embodiments, said personal care composition may comprise between about 0.05% to about 8% of at least one benefit agent encapsulated. In embodiments, said personal care composition may comprise between about 0.1% to about 5% of at least one encapsulated benefit agent.

In addition to capsules, personal care compositions of the present invention may also include additional ingredients.

Personal care compositions can be multi-phase or single phase. While the components for personal care compositions will be discussed below as being multi-phase for simplicity, the components for each phase could also be used in a single phase. A personal care composition can comprise a cleansing phase and a benefit phase. The cleansing phase and the benefit phase can be blended. The cleansing phase and the benefit phase can also be patterned (e.g. striped and/or marbled). In one aspect, the cleansing phase may comprise the capsules. I another aspect, the benefit phase may comprise the capsules.

### Cleansing Phase

A personal care composition can comprise from about 50% to about 99.5%, by weight of the composition, of a cleansing phase. A cleansing phase can include a surfactant. The personal care composition can further comprise from 2% to 20%, by weight of the rinse-off personal care composition, of a surfactant. Surfactants can comprise anionic surfactants, nonionic surfactants, amphoteric surfactants, zwitterionic surfactants, cationic surfactants, or mixtures thereof. The personal care composition can include at least one anionic surfactant. A personal care composition can also comprise, for example, an anionic surfactant, amphoteric surfactant, and a zwitterionic surfactant. Suitable amphoteric or zwitterionic surfactants, for example, can include those described in U.S. Pat. Nos. 5,104,646 and 5,106,609.

Anionic surfactants suitable for use in the cleansing phase of the present compositions include alkyl and alkyl ether sulfates. These materials have the respective formula ROSO₃M and RO(C₂H₄O)ₓSO₃M, wherein R is alkyl or alkenyl of from about 8 to about 24 carbon atoms, wherein x is about 1 to about 10, and M is a water-soluble cation such as ammonium, sodium, potassium, or triethanolamine. The alkyl ether sulfates are typically made as condensation products of ethylene oxide and monohydric alcohols having from about 8 to about 24 carbon atoms. R may have from about 10 to about 18 carbon atoms in both the alkyl and alkyl ether sulfates. The alcohols can be derived from fats, e.g., coconut oil or tallow, or can be synthetic. Lauryl alcohol and straight chain alcohols derived from coconut oil may be used. Such alcohols may be reacted with about 1 or about 3 to about 10 or about 5 molar proportions of ethylene oxide. The resulting mixture of molecular species may have, for example, an average of 3 moles of ethylene oxide per mole of alcohol, is sulfated and neutralized.

Specific examples of alkyl ether sulfates which may be used in the cleansing phase are sodium and ammonium salts of coconut alkyl triethylene glycol ether sulfate; tallow alkyl triethylene glycol ether sulfate, and tallow alkyl hexaoxyethylene sulfate. Suitable alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃—M], wherein R¹ is chosen from the group consisting of a straight or branched chain, saturated aliphatic hydrocarbon radical having from about 8 to about 24, or about 10 to about 18, carbon atoms; and M is a cation. Suitable examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₋₁₈ n-paraffins.

Suitable anionic surfactants for use in the cleansing phase include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, may be employed. Mixtures of anionic surfactants can also be used.

Amphoteric surfactants can include those that can be broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoactetate, disodium lauroamphoacetate disodium cocodiamphoacetate, and mixtures thereof. Amphoacetates and diamphoacetates can also be used.

Zwitterionic surfactants suitable for use as cleansing surfactant in the structured aqueous cleansing phase include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate.

Other zwitterionic surfactants suitable for use in the cleansing phase include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gammacarboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in the present compositions.

Amphoacetates and diamphoacetates can also be used. Non-limiting examples of suitable amphoacetates and diamphoacetates include sodium lauroamphoacetate, sodium cocoamphoactetate, disodium lauroamphoacetate, and disodium cocodiamphoacetate.

Cationic surfactants can also be used in the cleansing phase and may represent from 2% to about 5%, by weight of the cleansing phase.

Suitable nonionic surfactants for use in structured aqueous cleansing phase include condensation products of alkylene oxide groups (hydrophilic in nature) with an organic hydrophobic compound, which may be aliphatic or alkyl aromatic in nature.

Other suitable surfactants or cosurfactants that can generally be used in a cleansing phase for a rinse-off personal care composition are described in McCutcheon's: Detergents and Emulsifiers North American Edition (Allured Publishing Corporation 1947) (1986), McCutcheon's, Functional Materials North American Edition (Allured Publishing Corporation 1973) (1992) and U.S. Pat. No. 3,929,678 (filed Aug. 1, 1974).

The cleansing phase can include a structuring surfactant. Such a structuring surfactant can be included from 2% to about 20%, by weight of the personal care composition; from about 3% to about 15%, by weight of the personal care composition; or from about 5% to about 10%, by weight of the personal care composition. Such a structuring surfactant can include sodium trideceth(n) sulfate, hereinafter STnS, wherein n defines the average moles of ethoxylation. n can range, for example, from about 0 to about 3; n can range from about 0.5 to about 2.7; from about 1.1 to about 2.5; from about 1.8 to about 2.2; or n can be about 2. When n is less than 3, STnS can provide improved stability, improved compatibility of benefit agents within the rinse-off personal care compositions, and/or increased mildness of the rinse-off personal care compositions, such described benefits of STnS are disclosed in U.S. Patent Application Pub. No. 2012/0009285.

The personal care composition can further comprise from about 2% to 20%, by weight of the personal care composition, of a cosurfactant. Cosurfactants can comprise amphoteric surfactants, zwitterionic surfactants, or mixtures thereof. Examples of these types of surfactant are discussed above.

Personal care compositions can also comprise a water soluble cationic polymer. The water soluble cationic polymer can be present from about 0.001 to about 3 percent by weight of the personal care composition. The water soluble cationic polymer can also be present from about 0.05 to about 2 percent by weight of the personal care composition. The water soluble cationic polymer can also be present from about 0.1 to about 1 by weight of the personal care composition. The polymer may be in one or more phases as a deposition aid for the benefit agents described herein. Suitable cationic deposition polymers for use in the compositions of the present invention contain, for example, cationic nitrogen-containing moieties such as quaternary ammonium or cationic protonated amino moieties. The cationic protonated amines can be primary, secondary, or tertiary amines depending upon the particular species and the selected pH of the personal care composition.

Nonlimiting examples of cationic deposition polymers for use in compositions include polysaccharide polymers, such as cationic cellulose derivatives. The cationic cellulose polymers can be, for example, the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers. The water soluble cationic polymer comprises, for example, KG-30M. Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Ashland.

The water soluble cationic polymer can comprise, for example, a cationic guar. In one example, the cationic guar comprises guar hydroxypropyltrimonium chloride. The guar hydroxypropyltrimonium chloride can comprise, for example, N-hance™ CG-17 Cationic Guar. The cationic guar can be, for example, selected from a group consisting of N-hance™ 3196, Jaguar C-500, Jaguar C-17, and a combination thereof. Deposition polymers can have a cationic charge density from about 0.8 meq/g to about 2.0 meq/g or from about 1.0 meq/g to about 1.5 meq/g, or about 0.96 meq/g.

The water soluble cationic polymer can also comprise synthetic polyacrylamides. Examples of suitable synthetic polyacrylamides include polyquaternium 76 and Polymethylene-bis-acrylamide methacrylamido propyltrimethyl ammonium chloride (PAMMAPTAC, AM:MAPTAC ratio 88:12. In one example, the water soluble cationic polymer comprises PAM/MAPTAC.

A cleansing phase of a personal care composition can also include an associative polymer. Such associative polymer can be a crosslinked, alkali swellable, associative polymer comprising acidic monomers and associative monomers with hydrophobic end groups, whereby the associative polymer comprises a percentage hydrophobic modification and a hydrophobic side chain comprising alkyl functional groups. Without intending to be limited by theory, it is believed the acidic monomers can contribute to an ability of the associative polymer to swell in water upon neutralization of acidic groups; and associative monomers anchor the associative polymer into structured surfactant hydrophobic domains, e.g., lamellae, to confer structure to the surfactant phase and keep the associative polymer from collapsing and losing effectiveness in a presence of an electrolyte.

The crosslinked, associative polymer can comprise a percentage hydrophobic modification, which is a mole percentage of monomers expressed as a percentage of a total number of all monomers in a polymer backbone, including both acidic and other non-acidic monomers. Percentage hydrophobic modification of the associative polymer, hereafter % HM, can be determined by the ratio of monomers added during synthesis, or by analytical techniques such as proton nuclear magnetic resonance (NMR). Associative alkyl side chains can comprise, for example, butyl, propyl, stearyl, steareth, cetyl, lauryl, laureth, octyl, behenyl, beheneth, steareth, or other linear, branched, saturated, or unsaturated alkyl or alketh hydrocarbon side chains. The acidic monomer can comprise any acid functional group, for example sulfate, sulfonate, carboxylate, phosphonate, or phosphate or mixtures of acid groups. The acidic monomer can comprise, for example, a carboxylate, alternatively the acidic monomer is an acrylate, including acrylic acid and/or methacrylic acid. The acidic monomer comprises a polymerizable structure, e.g., vinyl functionality. Mixtures of acidic monomers, for example acrylic acid and methacrylic acid monomer mixtures, are useful.

The associative monomer can comprise a hydrophobic end group and a polymerizable component, e.g., vinyl, which can be attached. The hydrophobic end group can be attached to the polymerizable component, hence to the polymer chain, by different means but can be attached by an ether or ester or amide functionality, such as an alkyl acrylate or a vinyl alkanoate monomer. The hydrophobic end group can also be separated from the chain, for example, by an alkoxy ligand such as an alkyl ether. The associative monomer can be, for example, an alkyl ester, an alkyl (meth)acrylate, where (meth)acrylate is understood to mean either methyl acrylate or acrylate, or mixtures of the two.

The hydrophobic end group of the associative polymer can be incompatible with the aqueous phase of the composition and can associate with lathering surfactant hydrophobe components. Without intending to be limited by theory, it is believed that longer alkyl chains of structuring polymer hydrophobe end groups can increase incompatibility with the aqueous phase to enhance structure, whereas somewhat shorter alkyl chains having carbon numbers closely resembling lathering surfactant hydrophobes (e.g., 12 to 14 carbons) or multiples thereof (for bilayers, e.g.) can also be effective. An ideal range of hydrophobic end group carbon numbers combined with an optimal percentage of hydrophobic monomers expressed as a percentage of the polymer backbone can provide increased structure to the lathering, structured surfactant composition at low levels of polymer structurant.

The associative polymer can be Aqupec SER-300 made by Sumitomo Seika of Japan, which is Acrylates/C10-30 alkyl acrylate crosspolymer and comprises stearyl side chains with less than about 1% HM. Other preferred associative polymers can comprise stearyl, octyl, decyl and lauryl side chains. Preferred associative polymers are Aqupec SER-150 (acrylates/C10-30 alkyl acrylates crosspolymer) comprising about C18 (stearyl) side chains and about 0.4% HM, and Aqupec HV-701EDR which comprises about C8 (octyl) side chains and about 3.5% HM. In another example, the associative polymer can be Stabylen 30 manufactured by 3V Sigma S.p.A., which has branched isodecanoate hydrophobic associative side chains.

Other optional additives can be included in the cleansing phase, including for example an emulsifier (e.g., non-ionic emulsifier) and electrolytes. Suitable emulsifiers and electrolytes are described in U.S. patent application Ser. No. 13/157,665.

### Benefit Phase

As noted herein, personal care compositions can include a benefit phase. The composition may comprise from about 0.1% to about 50%, by weight of the composition, of a benefit phase. The benefit phase can be hydrophobic and/or anhydrous. The benefit phase can also be substantially free of or free of surfactant. In particular, the benefit phase can comprise from about 0.1% to about 50%, by weight of the rinse-off personal care composition, of a benefit agent. The benefit phase can include, for example, from about 0.5% to about 20%, by weight of the rinse-off personal care composition, of a skin conditioning agent as defined earlier. The skin conditioning agent is preferably selected from the group of benefit agents.

A benefit phase can have a particle size of about 4 to about 500 μm, from about 5 to about 300 μm, from about 6 to about 100 μm, or from about 10 to about 50 μm. The particle size is measured in neat product under a differential interference contrast optical microscope with a 10x objective lens. The particle size distribution is counted manually. All benefit phase particles are assumed as uniform spheres in this application. For irregular shaped benefit phase particles, the longest axis is used as the diameter for the particle size distribution counting. The number weighted average of all lipid particles is defined as the average lipid particle size. This measurement can also be accomplished with a computer algorithm.

A benefit phase can have a viscosity as measured by a standard rheometer, such as a Brookfield R/S plus. A sample of 2.5 mL is measured with a spindle C75-1 at a shear rate of 2 s¹ at 25° C. A benefit phase can generally have a viscosity of about 200 cP to about 15,000 cP.

However, it has been discovered that lower viscosity benefit phases (i.e. less than about 2000 cP) can be advantageous for manufacturing as it is easier to blend the benefit phase and the surfactant phase. Thus, for example, the benefit phase has a viscosity of 200 cP to about 1800 cP or from about 300 cP to about 1500 cP.

### Additional Ingredients

Additional ingredients can also be added to the personal care composition for treatment of the skin and/or hair, or to modify the aesthetics of the personal care composition as is the case with perfumes, colorants, dyes or the like. Materials useful in products herein can be categorized or described by their cosmetic and/or therapeutic benefit or their postulated mode of action or function. However, it can be understood that actives and other materials useful herein can, in some instances, provide more than one cosmetic and/or therapeutic benefit or function or operate via more than one mode of action. Therefore, classifications herein can be made for convenience and cannot be intended to limit an ingredient to particularly stated application or applications listed. A precise nature of these additional materials, and levels of incorporation thereof, will depend on the physical form of the composition and the nature of the cleansing operation for which it is to be used. The additional materials can usually be formulated at about 6% or less, about 5% or less, about 4% or less, about 3% or less, about 2% or less, about 1% or less, about 0.5% or less, about 0.25% or less, about 0.1% or less, about 0.01% or less, or about 0.005% or less of the rinse-off personal care composition.

To further improve stability under stressful conditions, such as high temperature and vibration, densities of separate phases can be adjusted such that they can be substantially equal. To achieve this, low density microspheres can be added to one or more phases of the rinse-off personal care composition. Examples of rinse-off personal care compositions that comprise low density microspheres are described in a patent application published on May 13, 2004 under U.S. Patent Publication No. 2004/0092415A1 entitled “Striped Liquid Personal Cleansing Compositions Containing A Cleansing Phase and A Separate Phase with Improved Stability,” filed on Oct. 31, 2003 by Focht, et al.

Other non-limiting ingredients that can be used in the personal care composition of the present invention can comprise an optional benefit component that can be selected from the group consisting of thickening agents; preservatives; antimicrobials; fragrances; chelators (e.g. such as those described in U.S. Pat. No. 5,487,884 issued to Bisset, et al.); sequestrants; vitamins (e.g. Retinol); vitamin derivatives (e.g. tocophenyl actetate, panthenol); sunscreens; desquamation actives (e.g. such as those described in U.S. Pat. Nos. 5,681,852 and 5,652,228 issued to Bisset); anti-wrinkle/anti-atrophy actives (e.g. N-acetyl derivatives, thiols, hydroxyl acids, phenol); anti-oxidants (e.g. ascorbic acid derivatives, tocophenol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); anti-acne medicaments; essential oils; sensates; pigments; colorants; pearlescent agents; interference pigments (e.g. such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A, entitled “Personal Care Compositions Containing Hydrophobically Modified Non-platelet particle filed on Feb. 15, 2005 by Taylor, et al.) and mixtures thereof. The multiphase personal care composition can comprise from about 0.1% to about 4%, by weight of the rinse-off personal care composition, of hydrophobically modified titanium dioxide. Other such suitable examples of such skin actives are described in U.S. patent application Ser. No. 13/157,665.

### Shave Care Composition

Capsules of the current invention can be used in shave compositions to provide one or more benefits, including freshness and/or cooling. The shave compositions of the present invention can be in different forms. Non-limiting examples of said forms are: shaving creams, shaving gels, aerosol shaving gels, shaving soaps, aerosol shaving foams, liquids, pastes, Newtonian or non-Newtonian fluids, gels, and sols.

The shave composition may comprise at least one benefit agent encapsulated in said capsules at a level where upon directed use, promotes one or more benefits. In one embodiment of the present invention, said shave composition comprises between about 0.01% to about 15% of at least one benefit agent encapsulated in said capsules. In another embodiment, said shave composition comprises between about 0.05% to about 8% of at least one benefit agent encapsulated. In another embodiment, said shave composition comprises between about 0.1% to about 5% of at least one benefit agent encapsulated.

In addition to at least one capsule, the shave compositions of the present invention may also include lathering surfactants, carriers, adjunct ingredients, and other additional ingredients.

### Lathering Surfactants

The shave compositions can comprise one or more lathering surfactants and a carrier such as water, at a total level of from about 60% to about 99.99%. A lathering surfactant defined herein as surfactant, which when combined with water and mechanically agitated generates a foam or lather. Preferably, these surfactants or combinations of surfactants should be mild, which means that these surfactants provide sufficient cleansing or detersive benefits but do not overly dry the skin or hair while still being able to produce a lather.

A wide variety of lathering surfactants are useful herein and include those selected from the group consisting of anionic lathering surfactants, nonionic lather surfactants, amphoteric lathering surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants.

Concentrations of these surfactant are from about 10% to about 20%, alternatively from about 5% to about 25%, and alternatively from 2% to about 60% by weight of the composition.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine). Also useful are taurates which are based on taurine, which is also known as 2-aminoethanesulfonic acid, and glutamates, especially those having carbon chains between C₈ and C₁₆.

Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Suitable amphoteric or zwitterionic detersive surfactants for use in the compositions herein include those which are known for use in hair care or other personal care cleansing. Concentration of such amphoteric detersive surfactants is from about 1% to about 10%, alternatively from about 0.5% to about 20% by weight of the composition. Non-limiting examples of suitable zwitterionic or amphoteric surfactants are described in U.S. Pat. Nos. 5,104,646 and 5,106,609.

Nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety. Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Lathering surfactants for use may be one or more of the following, wherein the anionic lathering surfactant is selected from the group consisting of ammonium lauroyl sarcosinate, sodium trideceth sulfate, sodium lauroyl sarcosinate, sodium myristoyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof; wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

One suitable lathering surfactant is a polyglyceryl fatty ester. In one embodiment the polyglyceryl fatty ester surfactant has the formula:

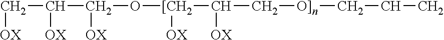

wherein n is 1 to 10, and X is a hydrogen atom or a long chain acyl group derived from a C₁₂₋₂₂ fatty acid or an N-fatty acyl-neutral amino acid, provided that at least one X is a long chain acyl group and no more than three X's are long chain acyl groups. In one embodiment, the polyglyceryl fatty ester surfactant is selected from the group consisting of: polyglyceryl-10 oleate, polyglyceryl-6 stearate, polyglyceryl-10 stearate, polyglyceryl-8 dipalmitate, polyglyceryl-10 dipalmitate, polyglyceryl-10 behenate, and polyglyceryl-12 trilaurate.

### Carriers

Shave compositions of the present invention can also comprise a carrier. In one embodiment the carrier comprises water. The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the shave composition comprises from about 50% to about 99.99%, preferably from about 60% to about 99.9%, more preferably from about 70% to about 98%, and even more preferably from about 80% to about 95% of the carrier by weight of the composition.

### Adjunct Ingredients

In embodiments, shave compositions may comprise at least one lubricant selected from: a lubricious water soluble polymer; a water insoluble particle, a hydrogel forming polymer, and a mixture thereof.

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Examples of suitable polyethylene oxides: PEG-23M, PEG-45M, and PEG-90M. The lubricious water soluble polymer can be at a level of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. Generally, the hydrogel-forming polymer will be included in the shaving composition in an amount of about 0.0005% to about 3%, or about 0.001% to about 0.5%, or about 0.002% to about 0.1%, by weight.

Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer. Glyceryl acrylate/acrylic acid copolymer is highly hydrophilic, has a molecular weight greater than 1 million daltons and generally includes a polyacrylic acid backbone partially esterified (typically about 50% esterified) with glycerin. It is believed that the glyceryl acrylate/acrylic acid copolymer forms a clathrate that holds water, which, upon release, supplies lubrication and moisturization to the skin. It has been found that shave gel compositions that include the glyceryl acrylate/acrylic acid copolymer have improved gel structure and reduced coefficient of friction (i.e., increased lubricity). See e.g. U.S. 2006/00257349 at ¶10.

The term “water dispersible”, as used herein, means that a substance is either substantially dispersible or soluble in water. The water dispersible surface active agent is preferably one that is capable of forming a lather, such as one or more of the optional lathering surfactants described in section 5 below (including but not limited to a soap, an interrupted soap, a detergent, an anionic surfactant, a non-ionic surfactant or a mixture of one or more of these.)

### 1. Polar Solvents

In embodiments, the carrier comprises a polar solvent. The level of polar solvent can be from about 1% to about 20%, or from about 5% to about 10%. Polar solvents useful herein include polyhydric alcohols such as, 3-butylene glycol, propane diol, ethylene glycol, diethylene glycol, sorbitol, and other sugars which are in liquid form at ambient temperature glycerin, sorbitol, propylene glycol, butylene glycol, pentylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof. Polyols such as those containing from 2 to about 6 carbon atoms and from 2 to about 6 hydroxy groups are preferred (e.g., 1,3-propanediol, ethylene glycol, glycerin, and 1,2-propanediol) can also be used. The most preferred are Butylene, Pentylene or Hexylene Glycol and mixtures thereof.

Without intending to be bound by theory, it is believed that the addition of one or more, polar solvents, allows for reduction in the viscosity and improvement in the clarity of the shave composition while maintaining good lubrication.

The shave composition of the present invention may comprise a salicylic acid compound, its esters, its salts, or combinations thereof. In the compositions of the present invention, the salicylic acid compound preferably comprises from about 0.1% to about 5%, preferably from about 0.2% to about 2%, and more preferably from about 0.5% to about 2%, by weight of the composition, of salicylic acid.

Shave compositions of the present invention may contain a variety of other ingredients that are conventionally used in given product types provided that they do not unacceptably alter the benefits of the invention. These ingredients should be included in a safe and effective amount for a shave composition for application to skin.

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof.

Additional non-limiting examples of additional suitable skin treatment actives are included in U.S. 2003/0082219 in Section I (i.e. hexamidine, zinc oxide); U.S. Pat. No. 5,665,339 at Section D (i.e. coolants, skin conditioning agents, sunscreens and pigments, and medicaments); and US 2005/0019356 (i.e. desquamation actives, anti-acne actives, chelators, flavonoids, and antimicrobial and antifungal actives). Other useful optional ingredients include: Anti-Wrinkle Actives and/or Anti-Atrophy Actives; Anti-Oxidants and/or Racial Scavengers; Anti-Inflammatory Agents; Anti-Cellulite Agents; Tanning Actives; Skin Lightening Agents; Sunscreen Actives; Water Soluble Vitamins; particulates; and combinations thereof.

The shave composition of the present invention is a non-aerosol composition. In one embodiment, the shave composition is free or substantially free of a volatile post-foaming agent.
a. Skin conditioning agents, more preferably a conditioning agent as defined earlier
b. Thickening Agents (including thickeners and gelling agents)

Compositions of the present invention can comprise one or more thickening agents, preferably from about 0.05% to about 10%, more preferably from about 0.1% to about 5%, and even more preferably from about 0.25% to about 4%, by weight of the composition. Nonlimiting classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); crosslinked polyacrylate polymers (including both cationic and nonionic polymers, such as described in U.S. Pat. Nos. 5,100,660; 4,849,484; 4,835,206; 4,628,078; 4,599,379, and EP 228,868); polymeric sulfonic acid (such as copolymers of acryloyldimethyltaurate and vinylpyrrolidone) and hydrophobically modified polymeric sulfonic acid (such as crosspolymers of acryloyldimethyltaurate and beheneth-25 methacrylate); polyacrylamide polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); polysaccharides (nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof); gums (i.e. gum agents such as acacia, agar, algin, alginic acid, ammonium alginate, amylopectin, calcium alginate, calcium carrageenan, camitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. No. 6,967,027 to Heux et al.; U.S. Pat. No. 5,207,826 to Westland et al.; U.S. Pat. No. 4,487,634 to Turbak et al.; U.S. Pat. No. 4,373,702 to Turbak et al. and 4,863,565 to Johnson et al., U.S. Patent Publ. No. 2007/0027108 to Yang et al.)

### Compositional pH

Shave compositions of the present invention preferably has a pH of less than about 9, more preferably less than about 7. In one embodiment the composition has a pH of less than about 5, or less than about 4. In one preferred embodiment the composition has a pH range of from about 2.5 to about 4.5. Suitable lathering surfactants for use at pH levels below about 4 can be selected from the group consisting of alkyl sulfonates, pareth sulfonates, sulfobetaines, alkylhydroxysultaines, alkylglucosides and mixtures thereof.

### Capsules

The liquid hair care, personal care and shave care compositions of the present disclosure further include a population of capsules. As described in more detail below, the capsules may include a core surrounded by substantially inorganic shell.

The capsules may be present in the composition in an amount that is from about 0.05% to about 20%, or from about 0.05% to about 10%, or from about 0.1% to about 5%, or from about 0.2% to about 2%, by weight of the composition. The composition may comprise a sufficient amount of capsules to provide from about 0.05% to about 10%, or from about 0.1% to about 5%, or from about 0.1% to about 2%, by weight of the composition, of perfume raw materials to the composition. When discussing herein the amount or weight percentage of the capsules, it is meant the sum of the shell material and the core material.

Capsules can have a mean shell thickness of about 10 nm to about 10,000 nm, preferably about 170 nm to about 1000 nm, more preferably about 300 nm to about 500 nm.

In embodiments capsules can have a mean volume weighted capsule diameter of about 0.1 micrometers to 300 micrometers, about 0.1 to about 200 micrometers, about 1 micrometers to about 200 micrometers, about 10 micrometers to about 200 micrometers, about 10 micrometers to about 50 micrometers. It has been advantageously found that large capsules (e.g., mean diameter of about 10 μm or greater) can be provided in accordance with embodiments herein without sacrificing the stability of the capsules as a whole and/or while maintaining good fracture strength.

It has surprisingly been found that in addition to the inorganic shell, the volumetric core-shell ratio can play an important role to ensure the physical integrity of the capsules. Shells that are too thin vs. the overall size of the capsule (core:shell ratio>98:2) tend to suffer from a lack of self-integrity. On the other hand, shells that are extremely thick vs. the diameter of the capsule (core:shell ratio<80:20) tend to have higher shell permeability in a surfactant-rich matrix. While one might intuitively think that a thick shell leads to lower shell permeability (since this parameter impacts the mean diffusion path of the active across the shell), it has surprisingly been found that the capsules of this invention that have a shell with a thickness above a threshold have higher shell permeability. It is believed that this upper threshold is, in part, dependent on the capsule diameter. Volumetric core-shell ratio is determined according to the method provided in the Test Method section below.

The capsules may have a volumetric core-shell ratio of 50:50 to 99:1, preferably from 60:40 to 99:1, preferably 70:30 to 98:2, more preferably 80:20 to 96:4.

It may be desirable to have particular combinations of these capsule characteristics. For example, the capsules can have a volumetric core-shell ratio of about 99:1 to about 50:50; and have a mean volume weighted capsule diameter of about 0.1 μm to about 200 μm, and a mean shell thickness of about 10 nm to about 10,000 nm. The capsules can have a volumetric core-shell ratio of about 99:1 to about 50:50; and have a mean volume weighted capsule diameter of about 10 μm to about 200 μm, and a mean shell thickness of about 170 nm to about 10,000 nm. The capsules can have a volumetric core-shell ratio of about 98:2 to about 70:30; and have a mean volume weighted capsule diameter of about 10 μm to about 100 μm, and a mean shell thickness of about 300 nm to about 1000 nm.

In certain embodiments, the mean volume weighted diameter of the capsule is between 1 and 200 micrometers, preferably between 1 and 10 micrometers, even more preferably between 2 and 8 micrometers. In another embodiment, the shell thickness is between 1 and 10000 nm, 1-1000 nm, 10-200 nm. In a further embodiment, the capsules have a mean volume weighted diameter between 1 and 10 micrometers and a shell thickness between 1 and 200 nm. It has been found that capsules with a mean volume weighted diameter between 1 and 10 micrometers and a shell thickness between 1 and 200 nm have a higher Fracture strength

Without intending to be bound by theory, it is believed that the higher Fracture strength provides a better survivability during the laundering process, wherein said process can cause premature rupture of mechanically weak capsules due to the mechanical constraints in the washing machine.

Capsules having a mean volume weighted diameter between 1 and 10 micrometers and a shell thickness between 10 and 200 nm, offer resistance to mechanical constraints only when made with a certain selection of the silica precursor used. In some embodiments, said precursor has a molecular weight between 2 and 5 kDa, even more preferably a molecular weight between 2.5 and 4 kDa. In addition, the concentration of the precursor needs to be carefully selected, wherein said concentration is between 20 and 60 w %, preferably between 40 and 60 w % of the oil phase used during the encapsulation.

Without intending to be bound by theory, it is believed that higher molecular weight precursors have a much slower migration time from the oil phase into the water phase. The slower migration time is believed to arise from the combination of three phenomenon: diffusion, partitioning, and reaction kinetics. This phenomenon is important in the context of small sized capsules, due to the fact that the overall surface area between oil and water in the system increases as the capsule diameter decreases. A higher surface area leads to higher migration of the precursor from the oil phase to the water phase, which in turn reduces the yield of polymerization at the interface. Therefore, the higher molecular weight precursor may be needed to mitigate the effects brought by an in increase in surface area, and to obtain capsules according to this invention.

Methods according to the present disclosure can produce capsule having a low coefficient of variation of capsule diameter. Control over the distribution of size of the capsules can beneficially allow for the population to have improved and more uniform fracture strength. A population of capsules can have a coefficient of variation of capsule diameter of 40% or less, preferably 30% or less, more preferably 20% or less.

For capsules containing a core material to perform and be cost-effective in consumer goods applications, such as liquid hair care, personal care and shave care compositions, they should: i) be resistant to core diffusion during the shelf life of the liquid product (e.g., low leakage or permeability); ii) have ability to deposit on the targeted surface during application (e.g. skin and hair) and iii) be able to release the core material by mechanical shell rupture at the right time and place to provide the intended benefit for the end consumer.

The capsules described herein can have an average fracture strength of 0.1 MPa to 10 MPa, preferably 0.25 MPa to 5 MPa, more preferably 0.25 MPa to 3 MPa. Fully inorganic capsules have traditionally had poor fracture strength, whereas for the capsules described herein, the fracture strength of the capsules can be greater than 0.25 MPa, providing for improved stability and a triggered release of the benefit agent upon a designated amount of rupture stress.

The core is oil-based. The core may be a liquid at the temperature at which it is utilized in a formulated product. The core may be a liquid at and around room temperature.

The core preferably includes a perfume raw material. The core may comprise from about 1 wt % to 100 wt % perfume, based on the total weight of the core. Preferably, the core can include 50 wt % to 100 wt % perfume based on the total weight of the core, more preferably 80 wt % to 100 wt % perfume based on the total weight of the core. Typically, higher levels of perfume are preferred for improved delivery efficiency.

The perfume raw material may comprise one or more, preferably two or more, perfume raw materials. The term “perfume raw material” (or “PRM”) as used herein refers to compounds having a molecular weight of at least about 100 g/mol and which are useful in imparting an odor, fragrance, essence, or scent, either alone or with other perfume raw materials. Typical PRMs comprise inter alia alcohols, ketones, aldehydes, esters, ethers, nitrites and alkenes, such as terpene. The PRMs may be characterized by their boiling points (B.P.) measured at the normal pressure (760 mm Hg), and their octanol/water partitioning coefficient (P), which may be described in terms of logP, determined according to the test method described in Test methods section. Based on these characteristics, the PRMs may be categorized as Quadrant I, Quadrant II, Quadrant III, or Quadrant IV perfumes, as described in more detail below. A perfume having a variety of PRMs from different quadrants may be desirable, for example, to provide fragrance benefits at different touchpoints during normal usage.

Perfume raw materials having a boiling point B.P. lower than about 250° C. and a logP lower than about 3 are known as Quadrant I perfume raw materials. Quadrant 1 perfume raw materials are preferably limited to less than 30% of the perfume composition. Perfume raw materials having a B.P. of greater than about 250° C. and a logP of greater than about 3 are known as Quadrant IV perfume raw materials, perfume raw materials having a B.P. of greater than about 250° C. and a logP lower than about 3 are known as Quadrant II perfume raw materials, perfume raw materials having a B.P. lower than about 250° C. and a logP greater than about 3 are known as a Quadrant III perfume raw materials.

Preferably the capsule comprises a perfume. Preferably, the perfume of the capsule comprises a mixture of at least 3, or even at least 5, or at least 7 perfume raw materials. The perfume of the capsule may comprise at least 10 or at least 15 perfume raw materials. A mixture of perfume raw materials may provide more complex and desirable aesthetics, and/or better perfume performance or longevity, for example at a variety of touchpoints. However, it may be desirable to limit the number of perfume raw materials in the perfume to reduce or limit formulation complexity and/or cost.

The perfume may comprise at least one perfume raw material that is naturally derived. Such components may be desirable for sustainability/environmental reasons. Naturally derived perfume raw materials may include natural extracts or essences, which may contain a mixture of PRMs. Such natural extracts or essences may include orange oil, lemon oil, rose extract, lavender, musk, patchouli, balsamic essence, sandalwood oil, pine oil, cedar, and the like.

The core may comprise, in addition to perfume raw materials, a pro-perfume, which can contribute to improved longevity of freshness benefits. Pro-perfumes may comprise nonvolatile materials that release or convert to a perfume material as a result of, e.g., simple hydrolysis, or may be pH-change-triggered pro-perfumes (e.g. triggered by a pH drop) or may be enzymatically releasable pro-perfumes, or light-triggered pro-perfumes. The pro-perfumes may exhibit varying release rates depending upon the pro-perfume chosen.

The core of the encapsulates of the present disclosure may comprise a core modifier, such as a partitioning modifier and/or a density modifier. The core may comprise, in addition to the perfume, from greater than 0% to 80%, preferably from greater than 0% to 50%, more preferably from greater than 0% to 30% based on total core weight, of a core modifier. The partitioning modifier may comprise a material selected from the group consisting of vegetable oil, modified vegetable oil, mono-, di-, and tri-esters of C₄-C₂₄ fatty acids, isopropyl myristate, dodecanophenone, lauryl laurate, methyl behenate, methyl laurate, methyl palmitate, methyl stearate, and mixtures thereof. The partitioning modifier may preferably comprise or consist of isopropyl myristate. The modified vegetable oil may be esterified and/or brominated. The modified vegetable oil may preferably comprise castor oil and/or soy bean oil.

The shell may comprise between 90% and 100%, preferably between 95% and 100%, more preferably between 99% and 100% by weight of the shell of an inorganic material. Preferably, the inorganic material in the shell comprises a material selected from metal oxide, semi-metal oxides, metals, minerals or mixtures thereof. Preferably, the inorganic material in the shell comprises materials selected from SiO₂, TiO₂, Al₂O₃, ZrO₂, ZnO₂, CaCO₃, Ca₂SiO₄, Fe₂O₃, Fe₃O₄, clay, gold, silver, iron, nickel, copper or a mixture thereof. More preferably, the inorganic material in the shell comprises a material selected from SiO₂, TiO₂, Al₂O₃, CaCO₃, or mixtures thereof, most preferably SiO₂.

The shell may include a first shell component. The shell may preferably include a second shell component that surrounds the first shell component. The first shell component can include a condensed layer formed from the condensation product of a precursor. As described in detail below, the precursor can include one or more precursor compounds. The first shell component can include a nanoparticle layer. The second shell component can include inorganic materials.

The inorganic shell can include a first shell component comprising a condensed layer surrounding the core and may further comprise a nanoparticle layer surrounding the condensed layer. The inorganic shell may further comprise a second shell component surrounding the first shell component. The first shell component comprises inorganic materials, preferably metal/semi-metal oxides, more preferably SiO2, TiO2 and Al2O3, or mixture thereof, and even more preferably SiO2. The second shell component comprises inorganic material, preferably comprising materials from the groups of Metal/semi-metal oxides, metals and minerals, more preferably materials chosen from the list of SiO₂, TiO₂, Al₂O₃, ZrO₂, ZnO₂, CaCO₃, Ca₂SiO₄, Fe₂O₃, Fe₃O₄, clay, gold, silver, iron, nickel, and copper, or mixture thereof, even more preferably chosen from SiO₂ and CaCO₃ or mixture thereof. Preferably, the second shell component material is of the same type of chemistry as the first shell component in order to maximize chemical compatibility.

The first shell component can include a condensed layer surrounding the core. The condensed layer can be the condensation product of one or more precursors. The one or more precursors may comprise at least one compound from the group consisting of Formula (I), Formula (II), or mixture thereof, wherein Formula (I) is (M^{v}O_{z}Yₙ)_{w}, and wherein Formula (II) is (M^{v}O_{z}YₙR¹ₚ)_{w}. It may be preferred that the precursor comprises only Formula (I) and is free of compounds according to Formula (II), for example so as to reduce the organic content of the capsule shell (i.e., no R¹ groups). Formulas (I) and (II) are described in more detail below.

The one or more precursors can be of Formula (I):

(M^{v}O_{z}Yₙ)_{w} (Formula I),

where M is one or more of silicon, titanium and aluminum, v is the valence number of M and is 3 or 4, z is from 0.5 to 1.6, preferably 0.5 to 1.5, each Y is independently selected from —OH, —OR², —NH₂, —NHR², —N(R²)₂, wherein R² is a C₁ to C₂₀ alkyl, C₁ to C₂₀ alkylene, C₆ to C₂₂ aryl, or a 5-12 membered heteroaryl comprising from 1 to 3 ring heteroatoms selected from O, N, and S, R³ is a H, C₁ to C₂₀ alkyl, C₁ to C₂₀ alkylene, C₆ to C₂₂ aryl, or a 5-12 membered heteroaryl comprising from 1 to 3 ring heteroatoms selected from O, N, and S, n is from 0.7 to (v-1), and w is from 2 to 2000.

The one or more precursors can be of Formula (I) where M is silicon. It may be that Y is —OR². It may be that n is 1 to 3. It may be preferable that Y is —OR² and n is 1 to 3. It may be that n is at least 2, one or more of Y is —OR², and one or more of Y is —OH.

R² may be C₁ to C₂₀ alkyl. R² may be C₆ to C₂₂ aryl. R² may be one or more of C₁ alkyl, C₂ alkyl, C₃ alkyl, C₄ alkyl, C₅ alkyl, C₆ alkyl, C₇ alkyl, and C₈ alkyl. R² may be C₁ alkyl. R² may be C₂ alkyl. R² may be C₃ alkyl. R² may be C₄ alkyl.

It may be that z is from 0.5 to 1.3, or from 0.5 to 1.1, 0.5 to 0.9, or from 0.7 to 1.5, or from 0.9 to 1.3, or from 0.7 to 1.3.

It may be preferred that M is silicon, v is 4, each Y is —OR², n is 2 and/or 3, and each R² is C₂ alkyl.

The precursor can include polyalkoxysilane (PAOS). The precursor can include polyalkoxysilane (PAOS) synthesized via a hydrolytic process.

The precursor can alternatively or further include one or more of a compound of Formula (II):

(M^{v}O_{z}YₙR¹ₚ)_{w} (Formula II),

where M is one or more of silicon, titanium and aluminum, v is the valence number of M and is 3 or 4, z is from 0.5 to 1.6, preferably 0.5 to 1.5, each Y is independently selected from —OH, —OR², —NH₂, —NHR², —N(R²)₂, wherein R² is selected from a C₁ to C₂₀ alkyl, C₁ to C₂₀ alkylene, C₆ to C₂₂ aryl, or a 5-12 membered heteroaryl comprising from 1 to 3 ring heteroatoms selected from O, N, and S, R³ is a H, C₁ to C₂₀ alkyl, C₁ to C₂₀ alkylene, C₆ to C₂₂ aryl, or a 5-12 membered heteroaryl comprising from 1 to 3 ring heteroatoms selected from O, N, and S; n is from 0 to (v-1); each R¹ is independently selected from the group consisting of: a C₁ to C₃₀ alkyl; a C₁ to C₃₀ alkylene; a C₁ to C₃₀ alkyl substituted with a member (e.g., one or more) selected from the group consisting of a halogen, —OCF₃, —NO₂, —CN, —NC, —OH, —OCN, —NCO, alkoxy, epoxy, amino, mercapto, acryloyl, —C(O)OH, —C(O)O-alkyl, —C(O)O-aryl, —C(O)O-heteroaryl, and mixtures thereof; and a C₁ to C₃₀ alkylene substituted with a member selected from the group consisting of a halogen, —OCF₃, —NO₂, —CN, —NC, —OH, —OCN, —NCO, alkoxy, epoxy, amino, mercapto, acryloyl, —C(O)OH, —C(O)O-alkyl, —C(O)O-aryl, and —C(O)O-heteroaryl; and p is a number that is greater than zero and is up to pmax, where pmax=60/[9*Mw(R¹)+8], where Mw(R¹) is the molecular weight of the R¹ group, and where w is from 2 to 2000.

R¹ may be a C₁ to C₃₀ alkyl substituted with one to four groups independently selected from a halogen, —OCF₃, —NO₂, —CN, —NC, —OH, —OCN, —NCO, alkoxy, epoxy, amino, mercapto, acryloyl, CO₂H (ie, C(O)OH), —C(O)O-alkyl, —C(O)O-aryl, and —C(O)O-heteroaryl. R¹ may be a C₁ to C₃₀ alkylene substituted with one to four groups independently selected from a halogen, —OCF₃, —NO₂, —CN, —NC, —OH, —OCN, —NCO, alkoxy, epoxy, amino, mercapto, acryloyl, CO₂H, —C(O)O-alkyl, —C(O)O-aryl, and —C(O)O-heteroaryl.

As indicated above, to reduce or even eliminate organic content in the first shell component, it may be preferred to reduce, or even eliminate, the presence of compounds according to Formula (II), which has R1 groups. The precursor, the condensed layer, the first shell component, and/or the shell may be free of compounds according to Formula (II).

The precursors of formula (I) and/or (II) may be characterized by one or more physical properties, namely a molecular weight (Mw), a degree of branching (DB) and a polydispersity index (PDI) of the molecular weight distribution. It is believed that selecting particular Mw and/or DB can be useful to obtain capsules that hold their mechanical integrity once left drying on a surface and that have low shell permeability in surfactant-based matrices. The precursors of formula (I) and (II) may be characterized as having a DB between 0 and 0.6, preferably between 0.1 and 0.5, more preferably between 0.19 and 0.4, and/or a Mw between 600 Da and 100000 Da, preferably between 700 Da and 60000 Da, more preferably between 1000 Da and 30000 Da. The characteristics provide useful properties of said precursor in order to obtain capsules of the present invention. The precursors of formula (I) and/or (II) can have a PDI between 1 and 50.

The condensed layer comprising metal/semi-metal oxides may be formed from the condensation product of a precursor comprising at least one compound of formula (I) and/or at least one compound of formula (II), optionally in combination with one or more monomeric precursors of metal/semi-metal oxides, wherein said metal/semi-metal oxides comprise TiO2, Al2O3 and SiO2, preferably SiO2. The monomeric precursors of metal/semi-metal oxides may include compounds of the formula M(Y)_{V-n}Rₙ wherein M, Y and R are defined as in formula (II), and n can be an integer between 0 and 3. The monomeric precursor of metal/semi-metal oxides may be preferably of the form where M is Silicon wherein the compound has the general formula Si(Y)₄₋ₙRₙ wherein Y and R are defined as for formula (II) and n can be an integer between 0 and 3. Examples of such monomers are TEOS (tetraethoxy orthosilicate), TMOS (tetramethoxy orthosilicate), TBOS (tetrabutoxy orthosilicate), triethoxymethylsilane (TEMS), diethoxy-dimethylsilane (DEDMS), trimethylethoxysilane (TMES), and tetraacetoxysilane (TAcS). These are not meant to be limiting the scope of monomers that can be used and it would be apparent to the person skilled in the art what are the suitable monomers that can be used in combination herein.

The first shell components can include an optional nanoparticle layer. The nanoparticle layer comprises nanoparticles. The nanoparticles of the nanoparticle layer can be one or more of SiO₂, TiO₂, Al₂O₃, ZrO₂, ZnO₂, CaCO₃, clay, silver, gold, and copper. Preferably, the nanoparticle layer can include SiO₂ nanoparticles.

The nanoparticles can have an average diameter between 1 nm and 500 nm, preferably between 50 nm and 400 nm.

The pore size of the capsules can be adjusted by varying the shape of the nanoparticles and/or by using a combination of different nanoparticle sizes. For example, non-spherical irregular nanoparticles can be used as they can have improved packing in forming the nanoparticle layer, which is believed to yield denser shell structures. This can be advantageous when limited permeability is required. The nanoparticles used can have more regular shapes, such as spherical. Any contemplated nanoparticle shape can be used herein.

The nanoparticles can be substantially free of hydrophobic modifications. The nanoparticles can be substantially free of organic compound modifications. The nanoparticles can include an organic compound modification. The nanoparticles can be hydrophilic.

The nanoparticles can include a surface modification such as but not limited to linear or branched C₁ to C₂₀ alkyl groups, surface amino groups, surface methacrylo groups, surface halogens, or surface thiols. These surface modifications are such that the nanoparticle surface can have covalently bound organic molecules on it. When it is disclosed in this document that inorganic nanoparticles are used, this is meant to include any or none of the aforementioned surface modifications without being explicitly called out.

The capsules of the present disclosure may be defined as comprising a substantially inorganic shell comprising a first shell component and a second shell component. By substantially inorganic it is meant that the first shell component can comprise up to 10 wt %, or up to 5 wt % of organic content, preferably up to 1 wt % of organic content, as defined later in the organic content calculation. It may be preferred that the first shell component, the second shell component, or both comprises no more than about 5 wt %, preferably no more than about 2 wt %, more preferably about Owt %, of organic content, by weight of the first or shell component.

While the first shell component is useful to build a mechanically robust scaffold or skeleton, it can also provide low shell permeability in liquid products containing surfactants such as laundry detergents, shower-gels, cleansers, etc. (see Surfactants in Consumer Products, J. Falbe, Springer-Verlag). The second shell component can greatly reduce the shell permeability which improves the capsule impermeability in surfactant-based matrices. A second shell component can also greatly improve capsule mechanical properties, such as a capsule rupture force and fracture strength. Without intending to be bound by theory, it is believed that a second shell component contributes to the densification of the overall shell by depositing a precursor in pores remaining in the first shell component. A second shell component also adds an extra inorganic layer onto the surface of the capsule. These improved shell permeabilities and mechanical properties provided by the 2^{nd} shell component only occur when used in combination with the first shell component as defined in this invention.

Capsules of the present disclosure may be formed by first admixing a hydrophobic material with any of the precursors of the condensed layer as defined above, thus forming the oil phase, wherein the oil phase can include an oil-based and/or oil-soluble precursor. Said precursor/hydrophobic material mixture is then used as a dispersed phase in conjunction with a water phase, where an O/W (oil-in-water) emulsion is formed once the two phases are mixed and homogenized via methods that are known to the person skilled in the art. Nanoparticles can be present in the water phase and/or the oil phase, irrespective of the type of emulsion that is desired. The oil phase can include an oil-based core modifier and/or an oil-based benefit agent and a precursor of the condensed layer. Suitable core materials to be used in the oil phase are described earlier in this document.

Once the emulsion is formed, the following steps may occur:
(a) the nanoparticles migrate to the oil/water interface, thus forming the nanoparticle layer.
(b) The precursor of the condensed layer comprising precursors of metal/semi-metal oxides will start undergoing a hydrolysis/condensation reaction with the water at the oil/water interface, thus forming the condensed layer surrounded by the nanoparticle layer. The precursors of the condensed layer can further react with the nanoparticles of the nanoparticle layer.

The precursor forming the condensed layer can be present in an amount between 1 wt % and 50 wt %, preferably between 10 wt % and 40 wt % based on the total weight of the oil phase.

The oil phase composition can include any compounds as defined in the core section above. The oil phase, prior to emulsification, can include between 10 wt % to about 99 wt % benefit agent.

In a method of making capsules according to the present disclosure, the oil phase may be the dispersed phase, and the continuous aqueous (or water) phase can include water, an acid or base, and nanoparticles. The aqueous (or water) phase may have a pH between 1 and 11, preferably between 1 and 7 at least at the time of admixing both the oil phase and the aqueous phase together. The acid can be a strong acid. The strong acid can include one or more of HCl, HNO₃, H₂SO₄, HBr, HI, HClO₄, and HClO₃, preferably HCl. The acid can be a weak acid. The weak acid can be acetic acid or HF. The concentration of the acid in the continuous aqueous phase can be between 10⁻⁷M and 5M. The base can be a mineral or organic base, preferably a mineral base. The mineral base can be a hydroxide, such as sodium hydroxide and ammonia. For example, the mineral base can be about 10⁻⁵ M to 0.01M NaOH, or about 10⁻⁵ M to about 1M ammonia. The list of acids and bases and their concentration ranges exemplified above is not meant to be limiting the scope of the invention, and other suitable acids and bases that allow for the control of the pH of the continuous phase are contemplated herein.

In a method of making the capsules according to the present disclosure, the pH can be varied throughout the process by the addition of an acid and/or a base. For example, the method can be initiated with an aqueous phase at an acidic or neutral pH and then a base can be added during the process to increase the pH. Alternatively, the method can be initiated with an aqueous phase at a basic or neutral pH and then an acid can be added during the process to decrease the pH. Still further, the method can be initiated with an aqueous phase at an acid or neutral pH and an acid can be added during the process to further reduce the pH. Yet further the method can be initiated with an aqueous phase at a basic or neutral pH and a base can be added during the process to further increase the pH. Any suitable pH shifts can be used. Further any suitable combinations of acids and bases can be used at any time in the method to achieve a desired pH. Any of the nanoparticles described above can be used in the aqueous phase. The nanoparticles can be present in an amount of about 0.01 wt % to about 10 wt % based on the total weight of the aqueous phase.

A method can include admixing the oil phase and the aqueous phase in a ratio of oil phase to aqueous phase of about 1:10 to about 1:1.

The second shell component can be formed by admixing capsules having the first shell component with a solution of second shell component precursor. The solution of second shell component precursor can include a water soluble or oil soluble second shell component precursor. The second shell component precursor can be one or more of a compound of formula (I) as defined above, tetraethoxysilane (TEOS), tetramethoxysilane (TMOS), tetrabutoxysilane (TBOS), triethoxymethylsilane (TEMS), diethoxy-dimethylsilane (DEDMS), trimethylethoxysilane (TMES), and tetraacetoxysilane (TAcS). The second shell component precursor can also include one or more of silane monomers of type Si(Y)₄₋ₙRₙ wherein Y is a hydrolysable group, R is a non-hydrolysable group, and n can be an integer between 0 and 3. Examples of such monomers are given earlier in this paragraph, and these are not meant to be limiting the scope of monomers that can be used. The second shell component precursor can include salts of silicate, titanate, aluminate, zirconate and/or zincate. The second shell component precursor can include carbonate and calcium salts. The second shell component precursor can include salts of iron, silver, copper, nickel, and/or gold. The second shell component precursor can include zinc, zirconium, silicon, titanium, and/or aluminum alkoxides. The second shell component precursor can include one or more of silicate salt solutions such as sodium silicates, silicon tetralkoxide solutions, iron sulfate salt and iron nitrate salt, titanium alkoxides solutions, aluminum trialkoxide solutions, zinc dialkoxide solutions, zirconium alkoxide solutions, calcium salt solution, carbonate salt solution. A second shell component comprising CaCO₃ can be obtained from a combined use of calcium salts and carbonate salts. A second shell component comprising CaCO₃ can be obtained from Calcium salts without addition of carbonate salts, via in-situ generation of carbonate ions from CO₂.

The second shell component precursor can include any suitable combination of any of the foregoing listed compounds.

The solution of second shell component precursor can be added dropwise to the capsules comprising a first shell component. The solution of second shell component precursor and the capsules can be mixed together between 1 minute and 24 hours. The solution of second shell component precursor and the capsules can be mixed together at room temperature or at elevated temperatures, such as 20° C. to 100° C.

The second shell component precursor solution can include the second shell component precursor in an amount between 1 wt % and 50 wt % based on the total weight of the solution of second shell component precursor.

Capsules with a first shell component can be admixed with the solution of the second shell component precursor at a pH of between 1 and 11. The solution of the second shell precursor can contain an acid and/or a base. The acid can be a strong acid. The strong acid can include one or more of HCl, HNO₃, H₂SO₄, HBr, HI, HClO₄, and HClO₃, preferably HCl. In other embodiments, the acid can be a weak acid. In embodiments, said weak acid can be acetic acid or HF. The concentration of the acid in the second shell component precursor solution can be between 10⁻⁷M and 5M. The base can be a mineral or organic base, preferably a mineral base. The mineral base can be a hydroxide, such as sodium hydroxide and ammonia. For example, the mineral base can be about 10⁻⁵ M to 0.01M NaOH, or about 10⁻⁵ M to about 1M ammonia. The list of acids and bases exemplified above is not meant to be limiting the scope of the invention, and other suitable acids and bases that allow for the control of the pH of the second shell component precursor solution are contemplated herein.

The process of forming a second shell component can include a change in pH during the process. For example, the process of forming a second shell component can be initiated at an acidic or neutral pH and then a base can be added during the process to increase the pH. Alternatively, the process of forming a second shell component can be initiated at a basic or neutral pH and then an acid can be added during the process to decrease the pH. Still further, the process of forming a second shell component can be initiated at an acid or neutral pH and an acid can be added during the process to further reduce the pH. Yet further the process of forming a second shell component can be initiated at a basic or neutral pH and a base can be added during the process to further increase the pH. Any suitable pH shifts can be used. Further any suitable combinations of acids and bases can be used at any time in the solution of second shell component precursor to achieve a desired pH. The process of forming a second shell component can include maintaining a stable pH during the process with a maximum deviation of +/−0.5 pH unit. For example, the process of forming a second shell component can be maintained at a basic, acidic or neutral pH. Alternatively, the process of forming a second shell component can be maintained at a specific pH range by controlling the pH using an acid or a base. Any suitable pH range can be used. Further any suitable combinations of acids and bases can be used at any time in the solution of second shell component precursor to keep a stable pH at a desirable range.

The emulsion can be cured under conditions to solidify the precursor thereby forming the shell surrounding the core.

The reaction temperature for curing can be increased to increase the rate at which solidified capsules are obtained. The curing process can induce condensation of the precursor. The curing process can be done at room temperature or above room temperature. The curing process can be done at temperatures 30° C. to 150° C., preferably 50° C. to 120° C., more preferably 80° C. to 100° C. The curing process can be done over any suitable period to enable the capsule shell to be strengthened via condensation of the precursor material. The curing process can be done over a period from 1 minute to 45 days, preferably 1 hour to 7 days, more preferably 1 hour to 24 hours. Capsules are considered cured when they no longer collapse. Determination of capsule collapse is detailed below. During the curing step, it is believed that hydrolysis of Y moieties (from formula (I) and/or (II)) occurs, followed by the subsequent condensation of a —OH group with either another —OH group or another moiety of type Y (where the 2 Y moieties are not necessarily the same). The hydrolysed precursor moieties will initially condense with the surface moieties of the nanoparticles (provided they contain such moieties). As the shell formation progresses, the precursor moieties will react with said preformed shell.

The emulsion can be cured such that the shell precursor undergoes condensation. The emulsion can be cured such that the shell precursor reacts with the nanoparticles to undergo condensation.

Shown below are examples of the hydrolysis and condensation steps described herein for silica-based shells:
Hydrolysis: ≡Si—OR+H₂O→≡Si—OH+ROH
Condensation: ≡Si—OH+≡Si—OR→≡Si—O—Si≡+ROH
   ≡Si—OH+≡Si—OH→≡Si—O—Si≡+H₂O.

For example, when a precursor of formula (I) or (II) is used, the following describes the hydrolysis and condensation steps:
Hydrolysis: ≡M—Y+H₂O→≡M—OH+YH
Condensation: ≡M—OH+≡M—Y→≡M—O—M≡+YH
   ≡M—OH+≡M—OH→≡M—O—M≡+H₂O.

The capsules may be provided as a slurry composition (or simply “slurry” herein). The result of the methods described herein may be a slurry containing the capsules. The slurry can be formulated into a product, such as a consumer product.

### Test Methods

Method to Determine logP

The value of the log of the Octanol/Water Partition Coefficient (logP) is computed for each PRM in the perfume mixture being tested. The logP of an individual PRM is calculated using the Consensus logP Computational Model, version 14.02 (Linux) available from Advanced Chemistry Development Inc. (ACD/Labs) (Toronto, Canada) to provide the unitless logP value. The ACD/Labs' Consensus logP Computational Model is part of the ACD/Labs model suite.

### Viscosity Method

The viscosity of neat product is determined using a Brookfield® DV-E rotational viscometer, spindle 2, at 60 rpm, at about 20-21° C.

### Mean Shell Thickness Measurement

The capsule shell, including the first shell component and the second shell component, when present, is measured in nanometers on twenty benefit agent containing delivery capsules making use of a Focused Ion Beam Scanning Electron Microscope (FIB-SEM; FEI Helios Nanolab 650) or equivalent. Samples are prepared by diluting a small volume of the liquid capsule dispersion (20 μl) with distilled water (1:10). The suspension is then deposited on an ethanol cleaned aluminium stub and transferred to a carbon coater (Leica EM ACE600 or equivalent). Samples are left to dry under vacuum in the coater (vacuum level: 10⁻⁵ mbar). Next 25-50 nm of carbon is flash deposited onto the sample to deposit a conductive carbon layer onto the surface. The aluminium stubs are then transferred to the FIB-SEM to prepare cross-sections of the capsules. Cross-sections are prepared by ion milling with 2.5 nA emission current at 30 kV accelerating voltage using the cross-section cleaning pattern. Images are acquired at 5.0 kV and 100 pA in immersion mode (dwell time approx. 10 ρs) with a magnification of approx. 10,000.

Images are acquired of the fractured shell in cross-sectional view from 20 benefit delivery capsules selected in a random manner which is unbiased by their size, to create a representative sample of the distribution of capsules sizes present. The shell thickness of each of the 20 capsules is measured using the calibrated microscope software at 3 different random locations, by drawing a measurement line perpendicular to the tangent of the outer surface of the capsule shell. The 60 independent thickness measurements are recorded and used to calculate the mean thickness.

Mean and Coefficient of Variation of Volume-Weighted Capsule Diameter

Capsule size distribution is determined via single-particle optical sensing (SPOS), also called optical particle counting (OPC), using the AccuSizer 780 AD instrument or equivalent and the accompanying software CW788 version 1.82 (Particle Sizing Systems, Santa Barbara, California, U.S.A.), or equivalent. The instrument is configured with the following conditions and selections: Flow Rate=1 mL/sec; Lower Size Threshold=0.50 μm; Sensor Model Number=LE400-05SE or equivalent; Auto-dilution=On; Collection time=60 sec; Number channels=512; Vessel fluid volume=50 ml; Max coincidence=9200. The measurement is initiated by putting the sensor into a cold state by flushing with water until background counts are less than 100. A sample of delivery capsules in suspension is introduced, and its density of capsules adjusted with DI water as necessary via autodilution to result in capsule counts of at most 9200 per mL. During a time period of 60 seconds the suspension is analyzed. The range of size used was from 1 μm to 493.3 μm.

### Volume Distribution:

$$CoVv ( \% ) = \frac{{σ}_{v}}{{μ}_{v}} * 100 \\ σ v = {\sum }_{i = 1 um}^{493.3 um} ( {x}_{i , v} * {( {d}_{i} - {μ}_{v} )}^{2} ) 0.5 \\ {μ}_{v} = \frac{{\sum }_{i = 1 um}^{493.3 um} ( {x}_{i , v} * {d}_{i} )}{{\sum }_{i = 1 um}^{493.3 um} {x}_{i , v}}$$

where:
CoVᵥ—Coefficient of variation of the volume weighted size distribution
σᵥ—Standard deviation of volume-weighted size distribution
μᵥ—mean of volume-weighted size distribution
dᵢ—diameter in fraction i
x_{i,v}—frequency in fraction i (corresponding to diameter i) of volume-weighted size distribution

$${x}_{i , v} = \frac{{x}_{i , n} * {d}_{i}^{3}}{{\sum }_{i = 1 um}^{493.3 um} ( {x}_{i , n} * {d}_{i}^{3} )}$$

### Volumetric Core-Shell Ratio Evaluation

The volumetric core-shell ratio values are determined as follows, which relies upon the mean shell thickness as measured by the Shell Thickness Test Method. The volumetric core-shell ratio of capsules where their mean shell thickness was measured is calculated by the following equation:

$$\frac{Core}{Shell} = \frac{{( 1 - \frac{2 * Thickness}{{D}_{caps}} )}^{3}}{( 1 - {( 1 - \frac{2 * Thickness}{{D}_{caps}} )}^{3} )}$$

wherein Thickness is the mean shell thickness of a population of capsules measured by FIBSEM and the D_{caps} is the mean volume weighted diameter of the population of capsules measured by optical particle counting.

This ratio can be translated to fractional core-shell ratio values by calculating the core weight percentage using the following equation:

$$\% Core = ( \frac{\frac{Core}{Shell}}{1 + \frac{Core}{Shell}} ) * 100$$

and shell percentage can be calculated based on the following equation:

% Shell=100−% Core.

### Degree of Branching Method

The degree of branching of the precursors was determined as follows: Degree of branching is measured using (29Si) Nuclear Magnetic Resonance Spectroscopy (NMR).

### Sample Preparation

Each sample is diluted to a 25% solution using deuterated benzene (Benzene-D6 “100%” (D, 99.96% available from Cambridge Isotope Laboratories Inc., Tewksbury, MA, or equivalent). 0.015M Chromium(III) acetylacetonate (99.99% purity, available from Sigma-Aldrich, St. Louis, MO, or equivalent) is added as a paramagnetic relaxation reagent. If glass NMR tubes (Wilmed-LabGlass, Vineland, NJ or equivalent) are used for analysis, a blank sample must also be prepared by filling an NMR tube with the same type of deuterated solvent used to dissolve the samples. The same glass tube must be used to analyze the blank and the sample.

### Sample Analysis

The degree of branching is determined using a Bruker 400 MHz Nuclear Magnetic Resonance Spectroscopy (NMR) instrument, or equivalent. A standard silicon (29Si) method (e.g. from Bruker) is used with default parameter settings with a minimum of 1000 scans and a relaxation time of 30 seconds.

### Sample Processing

The samples are stored and processed using system software appropriate for NMR spectroscopy such as MestReNova version 12.0.4-22023 (available from Mestrelab Research) or equivalent. Phase adjusting and background correction are applied. There is a large, broad, signal present that stretches from −70 to −136 ppm which is the result of using glass NMR tubes as well as glass present in the probe housing. This signal is suppressed by subtracting the spectra of the blank sample from the spectra of the synthesized sample provided that the same tube and the same method parameters are used to analyze the blank and the sample. To further account for any slight differences in data collection, tubes, etc., an area outside of the peaks of interest area should be integrated and normalized to a consistent value. For example, integrate −117 to −115 ppm and set the integration value to 4 for all blanks and samples.

The resulting spectra produces a maximum of five main peak areas. The first peak (Q0) corresponds to unreacted TAOS. The second set of peaks (Q1) corresponds to end groups. The next set of peaks (Q2) correspond to linear groups. The next set of broad peaks (Q3) are semi-dendritic units. The last set of broad peaks (Q4) are dendritic units. When PAOS and PBOS are analyzed, each group falls within a defined ppm range. Representative ranges are described in the following table:

| | # of Bridging Oxygen | |
|---|---|---|
| Group ID | per Silicon | ppm Range |
| Q0 | 0 | −80 to −84 |
| Q1 | 1 | −88 to −91 |
| Q2 | 2 | −93 to −98 |
| Q3 | 3 | −100 to −106 |
| Q4 | 4 | −108 to −115 |

Polymethoxysilane has a different chemical shift for Q0 and Q1, an overlapping signal for Q2, and an unchanged Q3 and Q4 as noted in the table below:

| | # of Bridging Oxygen | |
|---|---|---|
| Group ID | per Silicon | ppm Range |
| Q0 | 0 | −78 to −80 |
| Q1 | 1 | −85 to −88 |
| Q2 | 2 | −91 to −96 |
| Q3 | 3 | −100 to −106 |
| Q4 | 4 | −108 to −115 |

The ppm ranges indicated in the tables above may not apply to all monomers. Other monomers may cause altered chemical shifts, however, proper assignment of Q0-Q4 should not be affected.

Using MestReNova, each group of peaks is integrated, and the degree of branching can be calculated by the following equation:

$$Degree of Branching = ( 1 / 4 ) * \frac{3 * Q 3 + 4 * Q 4}{Q 1 + Q 2 + Q 3 + Q 4}$$

Molecular Weight and Polydispersity Index Determination Method

The molecular weight (Polystyrene equivalent Weight Average Molecular Weight (Mw)) and polydispersity index (Mw/Mn) of the condensed layer precursors described herein are determined using Size Exclusion Chromatography with Refractive Index detection. Mn is the number average molecular weight.

### Sample Preparation

Samples are weighed and then diluted with the solvent used in the instrument system to a targeted concentration of 10 mg/mL. For example, weigh 50 mg of polyalkoxysilane into a 5 mL volumetric flask, dissolve and dilute to volume with toluene. After the sample has dissolved in the solvent, it is passed through a 0.45 um nylon filter and loaded into the instrument autosampler.

### Sample Analysis

An HPLC system with autosampler (e.g. Waters 2695 HPLC Separation Module, Waters Corporation, Milford MA, or equivalent) connected to a refractive index detector (e.g. Wyatt 2414 refractive index detector, Santa Barbara, CA, or equivalent) is used for polymer analysis. Separation is performed on three columns, each 7.8 mm I.D.×300 mm in length, packed with 5 μm polystyrene-divinylbenzene media, connected in series, which have molecular weight cutoffs of 1, 10, and 60 kDA, respectively. Suitable columns are the TSKGel G1000HHR, G2000HHR, and G3000HHR columns (available from TOSOH Bioscience, King of Prussia, PA) or equivalent. A 6 mm I.D.×40 mm long 5 μm polystyrene-divinylbenzene guard column (e.g. TSKgel Guardcolumn HHR-L, TOSOH Bioscience, or equivalent) is used to protect the analytical columns. Toluene (HPLC grade or equivalent) is pumped isocratically at 1.0 mL/min, with both the column and detector maintained at 25° C. 100 μL of the prepared sample is injected for analysis. The sample data is stored and processed using software with GPC calculation capability (e.g. ASTRA Version 6.1.7.17 software, available from Wyatt Technologies, Santa Barbara, CA or equivalent.)

The system is calibrated using ten or more narrowly dispersed polystyrene standards (e.g. Standard ReadyCal Set, (e.g. Sigma Aldrich, PN 76552, or equivalent) that have known molecular weights, ranging from about 0.250-70 kDa and using a third order fit for the Mp verses Retention Time Curve.

Using the system software, calculate and report Weight Average Molecular Weight (Mw) and PolyDispersity Index (Mw/Mn).

### Method of Calculating Organic Content in First Shell Component

As used herein, the definition of organic moiety in the inorganic shell of the capsules according to the present disclosure is: any moiety X that cannot be cleaved from a metal precursor bearing a metal M (where M belongs to the group of metals and semi-metals, and X belongs to the group of non-metals) via hydrolysis of the M-X bond linking said moiety to the inorganic precursor of metal or semi-metal M and under specific reaction conditions, will be considered as organic. A minimal degree of hydrolysis of 1% when exposed to neutral pH distilled water for a duration of 24 h without stirring, is set as the reaction conditions.

This method allows one to calculate a theoretical organic content assuming full conversion of all hydrolysable groups. As such, it allows one to assess a theoretical percentage of organic for any mixture of silanes and the result is only indicative of this precursor mixture itself, not the actual organic content in the first shell component. Therefore, when a certain percentage of organic content for the first shell component is disclosed anywhere in this document, it is to be understood as containing any mixture of unhydrolyzed or pre-polymerized precursors that according to the below calculations give a theoretical organic content below the disclosed number.

### Example for Silane (but not Limited Thereto: See Generic Formulas at the End of the Document):

Consider a mixture of silanes, with a molar fraction Yi for each, and where i is an ID number for each silane. Said mixture can be represented as follows:

Si(XR)₁₋ₙRₙ

where XR is a hydrolysable group under conditions mentioned in the definition above, Rⁱₙᵢ is non-hydrolyzable under conditions mentioned above and nᵢ=0, 1, 2 or 3.

Such a mixture of silanes will lead to a shell with the following general formula:

$${SiO}_{\frac{( 4 - n )}{2}} {R}_{n}$$

Then, the weight percentage of organic moieties as defined earlier can be calculated as follows:
1) Find out Molar fraction of each precursor (nanoparticles included)
2) Determine general formula for each precursor (nanoparticles included)
3) Calculate general formula of precursor and nanoparticle mixture based on molar fractions
4) Transform into reacted silane (all hydrolysable groups to oxygen groups)
5) Calculate weight ratio of organic moieties vs. total mass (assuming 1 mole of Si for framework)

### EXAMPLE

| Raw | | Mw | weight | amount | Molar |
|---|---|---|---|---|---|
| material | Formula | (g/mol) | (g) | (mmol) | fraction |
| Sample AY | SiO(OEt)₂ | 134 | 1 | 7.46 | 0.57 |
| TEOS | Si(OEt)₄ | 208 | 0.2 | 0.96 | 0.07 |
| DEDMS | Si(OEt)₂Me₂ | 148.27 | 0.2 | 1.35 | 0.10 |
| SiO2 NP | SiO₂ | 60 | 0.2 | 3.33 | 0.25 |

To calculate the general formula for the mixture, each atoms index in the individual formulas is to be multiplied by their respective molar fractions. Then, for the mixture, a sum of the fractionated indexes is to be taken when similar ones occur (typically for ethoxy groups).

Note: Sum of all Si fractions will always add to 1 in the mixture general formula, by virtue of the calculation method (sum of all molar fractions for Si yields 1).

SiO_{1*0.57+2*0.25}(OEt)_{2*0.57+4*0.07+2*0.10}Me_{2*0.10}

SiO_{1.07}(OEt)_{1.62}Me_{0.20}

To transform the unreacted formula to a reacted one, simply divide the index of ALL hydrolysable groups by 2, and then add them together (with any pre-existing oxygen groups if applicable) to obtain the fully reacted silane.

SiO_{1.88} Me_{0.20}

In this case, the expected result is SiO_{1.9}Me_{0.20}, as the sum of all indexes must follow the following formula:

A+B/2=2,

where A is the oxygen atom index and B is the sum of all non-hydrolysable indexes. The small error occurs from rounding up during calculations and should be corrected. The index on the oxygen atom is then readjusted to satisfy this formula.

Therefore, the final formula is SiO_{1.9}Me_{0.2}, and the weight ratio of organic is calculated below:

Weight ratio=(0.20*15)/(28+1.9*16+0.20*15)=4.9%

### General Case:

The above formulas can be generalized by considering the valency of the metal or semi-metal M, thus giving the following modified formulas:

M(XR)_{V-ni}Rⁱₙᵢ

and using a similar method but considering the valency V for the respective metal.

### Leakage Method

The testing of capsule leakage in liquid compositions (e.g., shampoo, conditioners, body wash and skin care compositions, all of which will be referred to as formulation or matrices below) is performed as follows.

Homogenized slurry (of a known perfume activity, defined as the weight fraction of the perfume in the total slurry) is added and adequately dispersed to a known amount of as haircare of personal care composition base, such that the perfume weight fraction in the final formulation is of 0.25 w % (or between 0.2 w % and 0.3 w %).

The formulated product is stored in ajar or glass container covered with an airtight lid and where the volume of headspace above the liquid is no more than 5x the volume of the liquid itself, for 7 days at 35C and 40% relative humidity.

### Sample Preparation

After the 7 days of storage, samples of capsules, total oil, and free oil are prepared as follows:

(a) Preparation of capsule sample: Between 0.1 g and 0.11 g of the formulation containing slurry is introduced into the bottom of a GC vial (see below for specific of the GC vials and method) and where the GC vials are capped with a crimp cap to yield an airtight milieu, thus obtaining the capsule sample. This step is performed twice to obtain two readings, and the mean of the two values will be used, provided they do not differ too much from each other, in which case the analysis needs to be repeated. The GC vials are then analyzed via GC/MS, as detailed below.

(b) Preparation of total oil sample: A 1 gram aliquot of the formulation is introduced into a 7 ml cylindrical shape vial of a diameter of 1 cm to 1.5 cm, equipped with a magnetic stirring bar of length no less than the radius of the 7 ml vial, thus ensuring proper mixing in the vial. The 1 gram aliquot in the 7 ml vial is then mixed on a stirring plate for 24 h at 500 rpm, thereby ensuring that the capsules are broken by the grinding action of the stir bar against the bottom of the 7 ml vial. Optical microscopy can be used to verify that no more intact capsules remain. In case such capsules are found, the step is repeated for an additional 24 h, or until all or almost all capsules are broken. Then, the formulation containing broken capsules is introduced into GC vials in a similar manner as for step (a). This yields total oil samples. It is to be noted that the capsule sample and the total oil sample are not analyzed on the same day, as there is a need to prepare the total oil sample after the leakage sample has been removed from storage. It is to be noted that the capsule sample and the total oil sample are not analyzed on the same day, as there is a need to prepare the total oil sample after the capsule sample has been removed from storage. This does not affect (or does not substantially affect) the results.

(c) Preparation of free oil sample: A beauty care formulation containing between 0.2 w % and 0.3 w % (preferably 0.25 w %) of free oil is prepared, by adding and adequately dispersing a known amount of a perfume oil composition into a known amount of beauty care base. The perfume oil composition formulated herein is representative of the perfume oil composition that is present in the slurry. Then the free oil formulation is introduced into GC vials in a similar manner as for step (a). This yields reference samples, which must be used when analyzing both the capsule sample and the total oil sample.

On each day of analysis, the capsule samples or total oil samples must be run in conjunction with the reference sample.

### GC/MS Method

For each sample, test and reference, aliquots of 0.1 gr to 0.11 gr of sample are transferred to 20 ml headspace vials (Gerstel SPME vial 20 ml, part no. 093640-035-00) and immediately sealed (sealed with Gerstel Crimp caps for SPME, part no. 093640-050-00). Two headspace vials are prepared for each sample. The sealed headspace vials are then allowed to equilibrate. Samples reach equilibrium after 3 hours at room temperature, but can be left to sit longer without detriment or change to the results, up until 24 hours after sealing the headspace vial. After equilibrating, the samples are analyzed by GC/MS.

GS/MS analysis are performed by sampling the headspace of each vial via SPME (50/30 μm DVB/Carboxen/PDMS, Sigma-Aldrich part #57329-U), with a vial penetration of 25 millimeters and an extraction time of 1 minute at room temperature. The SPME fiber is subsequently on-line thermally desorbed into the GC injector (270° C., splitless mode, 0.75 mm SPME Inlet liner (Restek, art #23434) or equivalent, 300 seconds desorption time and injector penetration of 43 millimeters). The perfume composition is analyzed by fast GC/MS in full scan mode. Ion extraction of the specific mass for each component is obtained.

### Leakage Calculations

The leakage is calculated as follows, separately for the capsule sample and total oil sample, where “Area” denotes the area under the chromatogram peak corresponding to the PRM of interest:

For each PRM, the following formula gives a PRM leakage:

$$PRM leakage = \frac{{Area}_{PRM Capsule ( or total oil ) sample}}{{Area}_{PRM reference sample}}$$

Once calculated for all PRMs for both the total oil sample and the capsule sample, the corrected PRM leakage can be calculated using the following formula:

$$Corrected PRM leakage = \frac{PRM {leakage}_{capsule sample}}{PRM {leakage}_{total oil sample}}$$

Once the corrected PRM leakage has been calculated for all PRMs, the Average leakage can be found by taking the arithmetic mean of each corrected PRM leakage.

Perfume Composition

TABLE 1
Composition of Perfume 1
Perfume 1

| Perfume Raw Material | CAS # | logP |
|---|---|---|
| Ethyl 2-methyl butyrate | 7452-79-1 | 2.16 |
| Eucalyptol | 470-82-6 | 2.74 |
| 2,4-dimethylcyclohex-3-ene-1- | 68039-49-6 | 2.34 |
| carbaldehyde | | |
| Tetrahydro myrcenol | 18479-57-7 | 3.54 |
| Tetrahydro linalool | 78-69-3 | 3.48 |
| iso-Bornyl acetate | 125-12-2 | 3.60 |
| (2-tert-butylcyclohexyl) acetate | 88-41-5 | 4.23 |
| (4-tert-butylcyclohexyl) acetate | 32210-23-4 | 4.23 |
| Verdyl acetate | 5413-60-5 | 3.63 |
| beta-Naphthyl methyl ether | 93-04-9 | 3.47 |

### Hair Care Compositions

TABLE 2
Hair Care Composition 1
Hair Care composition 1

| Perfume Raw Material | CAS # |
|---|---|
| Water | |
| Natrasol 250 HHW 1,300 kDa | 9004-62-0 |
| (Hydroxyethylcellulose from | |
| Ashland) | |
| Polyethylene Glycol, 2,000 kDa | 25322-68-3 |
| Quaternium-18 | 61789-80-8 |
| Stearamidopropyl dimethylamine | 7651-02-7 |
| Cetearyl alcohol | 67762-27-0 |
| Cetyl alcohol | 36653-82-4 |
| Stearyl alcohol | 112-92-5 |
| Glyceryl stearate | 31566-31-1 |
| Oleyl alcohol | 143-28-2 |
| Methyl paraben | 99-76-3 |
| Propyl paraben | 94-13-3 |
| Benzyl alcohol | 100-51-6 |
| Citric acid | 77-92-9 |
| Phenoxyethanol | 122-99-6 |

TABLE 3
Hair Care Composition 2
Hair Care composition 2

| Perfume Raw Material | CAS # |
|---|---|
| Water | |
| Sodium Laureth Sulfate | 9004-82-4 |
| Cocamidopropyl betaine | 61789-40-0 |
| Tetrasodium EDTA | 64-02-8 |
| Sodium Benzoate | 532-32-1 |
| glycerin | 56-81-5 |
| NHance 3196 (available from | 65497-29-2 |
| Ashland) | |
| UCARE ™ JR-30M (Polyquaternium- | 81859-24-7 |
| 10 available from DOW) | |
| Flocare C106 (Polyquaternium-6 | 26062-79-3 |
| available from SNF) | |
| Cetyl alcohol | 36653-82-4 |
| Stearyl alcohol | 112-92-5 |

Unless mentioned otherwise, the chemicals from above table are available from Sigma Aldrich

TABLE 4
Hair Care Composition 3
Hair Care composition 3

| Perfume Raw Material | CAS # |
|---|---|
| Water | |
| Disodium EDTA | 139-33-3 |
| Stearyl alcohol | 112-92-5 |
| Cetyl alcohol | 36653-82-4 |
| Dicetyldimonium chloride (available | 1812-53-9 |
| from ABCR chemicals) | |
| Behentrimonium methosulfate | 81646-13-1 |
| (available from Alfa chemistry) | |
| Benzyl alcohol | 100-51-6 |
| Methylchloroisothiazoline | 26172-55-4 |

Unless mentioned otherwise, the chemicals from above table are available from Sigma Aldrich

### Personal Care Compositions

TABLE 5
Prophetic Personal Care Composition 1
Personal care composition 1

| Perfume Raw Material | CAS # |
|---|---|
| Water | |
| Sodium Trideceth Sulfate ((sulfated | 25446-78-0 |
| from trideceth-2,Stepan) | |
| Cocoamidopropyl Betaine | 61789-40-0 |
| Trideceth-3 (available from Alfa | 4403-12-7 |
| chemistry) | |
| Sodium Chloride | 7647-14-5 |
| Guar hydroxypropyltrimonium | 65497-29-2 |
| chloride (N-Hance CG-17 from | |
| Aqualou) | |
| Xanthan Gum (Keltrol 1000 from CP | 11138-66-2 |
| Kelco) | |
| Acrylates/C10-30 Alkylacrylate cross | N/A |
| polymer (aqupec SER-300C from | |
| sumitomo | |
| Methyl chloroisothiazoline | 26172-55-4 |
| EDTA | 60-00-4 |
| Sodium Benzoate | 532-32-1 |
| Perfume | N/A |
| RBD soybean oil (available from | 8000-22-7 |
| avatar corp) | |
| Glyceryl oleate | 82005-46-7 |
| Petrolatum | 8009-03-8 |
| Citric acid | 77-92-9 |

Unless mentioned otherwise, the chemicals from above table are available from Sigma Aldrich

### Synthesis of Silica Shell Based Perfume Capsules

The oil phase was prepared by mixing and homogenizing 2 gr of a non-hydrolytic precursor (see below) with 4 gr of perfume 1.

The water phase was prepared by adding 5 gr of Aerosil 300 (available from Evonik) to 195 gr of 0.1M HCl (available from Sigma Aldrich) in a glass vessel, after which the mixture was dispersed with an IKA S25N-25F Ultraturrax rotor-stator at 15000 rpm during 15 minutes. The solution was let cooling to room temperature before usage in case of heat generation during the dispersion.

Once each phase was prepared separately, 16 gr of the above water phase was added to the entirety of the prepared oil phase, and the oil phase was dispersed into the water phase with IKA ultraturrax S25N-10G mixing tool at 13400 RPM per 2 minute which formed an oil-in-water emulsion. Once the emulsification step was completed, the resulting emulsion was cured with the following temperature profile without stirring: 4 h at 30° C. and 16 h at 90° C., which yielded capsules of the present invention comprising a first shell component surrounding perfume 1.

To deposit a second shell component, 10 gr of the above capsules were added into 12 gr of Demineralized water and the resulting mixture was trimmed with 1M NaOH until the pH reached 7. Next, 2 ml of a 10 w % solution of sodium metasilicate (available from sigma Aldrich) in demineralized water solution was added at a rate of 20 microliters/min with a syringe pump, whilst maintaining the pH between 6.5 and 7.5 with a simultaneous addition of 1.6M HCl (Aq.) and continuous mixing at 400 rpm with an overhead mixer.

After the infusion of the second shell component solution had finished, the capsules were centrifuged for 10 minutes at 2500 RPM and re-dispersed in de-ionized water.

### Non-Hydrolytic Precursor Synthesis

1200 g of tetraethoxysilane (TEOS, available from Sigma Aldrich) was added to a clean dry round bottom flask equipped with a stir bar and distillation apparatus with a vigreux column under nitrogen atmosphere. 588 gr of acetic anhydride (available from Sigma Aldrich) and 7 g of Tetrakis(trimethylsiloxy)titanium (available from Gelest) were added and the contents of the flask were stirred for 16 hours at 135° C. During this time, the ethyl acetate generated by reaction of the ethoxy silane groups with acetic anhydride was distilled off. The reaction flask was cooled to room temperature and was placed on a rotary evaporator (Buchi Rotovapor R110), used in conjunction with a water bath and vacuum pump (Welch 1402 DuoSeal) to remove any remaining solvent and volatile compounds. The polyethoxysilane (PEOS) generated was a yellow viscous liquid with the following specifications found in TABLE 6 below. The ratio of TEOS to acetic anhydride can be varied to control the parameters presented in TABLE 6.

Specifications of the PEOS used for the making of the silica shell capsules of this invention.

TABLE 6

| Parameters of PEOS | Results |
|---|---|
| Degree of branching (DB) | 0.26 |
| Molecular weight (Mw) | 1.63 |
| Polydispersity index (PDI) | 1.81 |

### Non-Hydrolytic PEOS Synthesis:

1000 gr of TEOS (available from Sigma Aldrich) was added to a clean dry round bottom flask equipped with a stir bar and distillation apparatus under nitrogen atmosphere. Next, 564 gr of acetic anhydride (available from Sigma Aldrich) and 5.9 gr of Tetrakis(trimethylsiloxide) titanium (available from Gelest, Sigma Aldrich) were added and the contents of the flask and heated to 135C under stirring. The reaction temperature was maintained at 135C under vigorous stirring for 30 hours, during which the organic ester generated by reaction of the alkoxy silane groups with acetic anhydride was distilled off along with additional organic esters generated by the condensation of silyl-acetate groups with other alkoxysilane groups which occurred as the polyethoxysilane (PEOS) was generated. The reaction flask was cooled to room temperature and placed on a rotary evaporator (Buchi Rotovapor R110), used in conjunction with a water bath and vacuum pump (Welch 1402 DuoSeal) to remove any remaining solvent. The degree of branching (DB), Molecular weight (Mw) and polydispersity index (PDI) of the PEOS polymer synthetized were respectively 0.42, 2.99 and 2.70.

### Capsule Synthesis:

[Five batches were made following the procedure below, and after the curing step, the 5 batches were combined to yield a combined slurry:

The oil phase was prepared by mixing and homogenizing (or even dissolving if all compounds are miscible) 3 g of the PEOS precursor synthesized above with 2 g of a benefit agent and/or a core modifier, here a fragrance oil. 100 gr of water phase was prepared by mixing 0.5 g of NaCl, 3.5 gr of Aerosil 300 fumed silica from Evonik and 96 gr of DI water. The fumed silica was dispersed in the aqueous phase with an IKA ultra-turrax (S25N) at 20000 RPM for 15 min.

Once each phase was prepared separately, 5 g of the oil phase was dispersed into 16 g of the water phase with an IKA Ultra-Turrax mixer (S25N-10 g) at 25000 RPM for 5 minutes to reach a desired mean oil droplet diameter. Then the pH was brought to 1 using HCl 0.1M added dropwise. Once the emulsification step was complete, the resulting emulsion was left resting without stirring for 4 hours at room temperature, and then 16 hours at 90° C. until enough curing had occurred for the capsules to not collapse. The five batches were combined after the curing step, to obtain a combined capsule slurry.

In order to deposit a second shell component, the combined capsule slurry received a post-treatment with a second shell component solution. 50 g of the combined slurry was diluted with 50 g of 0.1M HCl(aq). The pH was adjusted to 7 using 1M NaOH(aq) added dropwise. Then, the diluted slurry was treated with a controlled addition (40 μl per minute) of the second shell component precursor solution (20 ml of 15 w % of Sodium silicate(aq.)), using a suspended magnetic stirrer reactor at 300 RPM, at room temperature. The pH was kept constant at pH 7 by continuously infusing 1.6M HCl(aq) and 1M NaOH(aq) solutions. Then the capsules were centrifuged per 10 minutes at 2500 RPM. The supernatant was discarded, and the capsules were re-dispersed in de-ionized water.

To test whether capsules collapse, the slurry was diluted 10 times into de-ionized water. Drops of the subsequent dilution were added to a microscopy microslide and left to dry overnight at room temperature. The following day, the dried capsules were observed under an optical microscope by light transmission to assess if the capsules have retained their spherical shape (without the use of a cover slide). The capsules survived drying and didn't collapse. The mean volume weighted diameter of the capsules measured was 5.3 μm with a CoV of 46.2%. The percentage of organic content in the shell was 0%.

### Polyacrylate Shell Based Perfume Capsules

A population of perfume capsules comprising a polyacrylate shell, encapsulating the same perfume composition as the silica shell based perfume capsules above, was prepared according to encapsulates made according to the processes disclosed in US Publication No. 2011/0268802

Formulation into Products

The capsule slurries synthetized above were combined with hair cair compositions 1 to 3 from tables X to Y respectively within 50 ml falcon tubes (for quantities to use, see leakage test method above). The capsules were dispersed with a SpeedMixer (Hauschild) at 1200 rpm for 3 minutes, and 1100 rpm for 1 min, after which the capsules and perfumes were well incorporated into the matrices. The homogeneous incorporation of capsules was verified by sampling small aliquots from 3 different locations within the products and observing via optical microscopy that there were similar quantities of capsules present in each of the 3 locations.

This yielded 6 hair care compositions containing capsules:
Hair care compositions 1 to 3+silica shell capsules (examples of the present invention)
Hair care compositions 1 to 3+polyacrylate shell capsules (comparative examples)

The above formulated products were then analyzed via the leakage method described in the test methods section.

### Results

Leakage results per PRM for Silica shell capsules and polyacrylate shell capsules in hair care compositions 1 and 2.

TABLE 7

| | | | Leakage (as %) after 1 week at 35° C. | | | |
|---|---|---|---|---|---|---|
| | | | Silica shell | Silica shell | Polyacrylate | Polyacrylate |
| | | | capsule + | capsule + | shell + | shell + |
| Perfume 1 | | | hair care | hair care | product | product |
| Perfume Raw | | | composition | composition | composition | composition |
| Material | CAS # | logP | 1 | 2 | 1 | 2 |
| Ethyl 2-methyl | 7452-79-1 | 2.16 | 11% | 18% | 56% | 56% |
| butyrate | | | | | | |
| Eucalyptol | 470-82-6 | 2.74 | 11% | 13% | 1% | 1% |
| 2,4- | 68039-49-6 | 2.34 | 10% | 21% | 62% | 62% |
| dimethylcyclohex- | | | | | | |
| 3-ene-1- | | | | | | |
| carbaldehyde | | | | | | |
| Tetrahydro | 18479-57-7 | 3.54 | 14% | 8% | 12% | 12% |
| myrcenol | | | | | | |
| Tetrahydro linalool | 78-69-3 | 3.48 | 9% | 7% | 10% | 10% |
| iso-Bornyl acetate | 125-12-2 | 3.60 | 22% | 6% | 0% | 0% |
| (2-tert- | 88-41-5 | 4.23 | 27% | 5% | 1% | 1% |
| butylcyclohexy1) | | | | | | |
| acetate | | | | | | |
| (4-tert- | 32210-23-4 | 4.23 | 17% | 5% | 1% | 1% |
| butylcyclohexyl) | | | | | | |
| acetate | | | | | | |
| Verdyl acetate | 5413-60-5 | 3.63 | 12% | 9% | 6% | 6% |
| beta-Naphthyl | 93-04-9 | 3.47 | 23% | 23% | 52% | 52% |
| methyl ether | | | | | | |
| | | Average: | 15.6% | 11.5% | 20.1% | 20.1% |
| | | StdDev: | 6.3% | 6.8% | 25.7% | 25.6% |

Leakage results per PRM for Silica shell capsules and polyacrylate shell capsules in hair care composition 3.

TABLE 8

| | | | Leakage (as %) after 1 | |
|---|---|---|---|---|
| | | | week at 35° C. | |
| | | | Silica | Polyacrylate |
| | | | shell + | shell + |
| Perfume 1 | | | product | product |
| Perfume Raw | | | composition | composition |
| Material | CAS # | logP | 3 | 3 |
| Ethyl 2-methyl | 7452-79-1 | 2.16 | 25% | 86% |
| butyrate | | | | |
| Eucalyptol | 470-82-6 | 2.74 | 21% | 1% |
| 2,4-dimethyl- | 68039-49- | 2.34 | 23% | 64% |
| cyclohex- | 6 | | | |
| 3-ene-1- | | | | |
| carbaldehyde | | | | |
| Tetrahydro | 18479-57- | 3.54 | 21% | 13% |
| myrcenol | 7 | | | |
| Tetrahydro | 78-69-3 | 3.48 | 21% | 11% |
| linalool | | | | |
| iso-Bornyl | 125-12-2 | 3.60 | 22% | 0% |
| acetate | | | | |
| (2-tert- | 88-41-5 | 4.23 | 23% | 1% |
| butylcyclohexyl) | | | | |
| acetate | | | | |
| (4-tert- | 32210-23- | 4.23 | 18% | 2% |
| butylcyclohexyl) | 4 | | | |
| acetate | | | | |
| Verdyl acetate | 5413-60-5 | 3.63 | 19% | 5% |
| beta-Naphthyl | 93-04-9 | 3.47 | 20% | 7% |
| methyl ether | | | | |
| | | Average: | 21.3% | 25.4% |
| | | StdDev: | 2% | 25.6% |

The leakage results from TABLES 7 and 8 show 2 aspects: First, that the average leakage of capsules of this invention (i.e. silica shell capsules) is the same or lower than the average leakage of comparative capsules (polyacrylate shell capsules). Secondly, the leakage of the different PRMs of perfume 1 (composition in table 1) are very similar to each other for capsules of this invention (i.e. silica shell capsules) as opposed to the comparative capsules (i.e. polyacrylate shell capsules). This shows that the silica shell capsules of this invention have a very uniform leakage as the standard deviation of the leakage of the different PRMs is low compared to comparative capsules. The fact that all PRMs leak at a similar rate allows for a more consistent freshness experience for consumers, whereas products where the PRMs leak at different rates will not have a consistent freshness character across the lifetime of the consumer goods.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A haircare composition compromising:
a surfactant;
at least one of a fatty alcohol, cationic polymer, or a mixture thereof;
one or more capsules;
a capsule comprising a core and a shell surrounding the core;
wherein the core comprises perfume raw materials;
wherein the shell comprises—
a substantially inorganic first shell component comprising a condensed layer and a nanoparticle layer;
wherein the condensed layer comprises a condensation product of a precursor;
wherein the nanoparticle layer comprises inorganic nanoparticles; and
wherein the condensed layer is disposed between the core and the nanoparticle layer;
an inorganic second shell component surrounding the first shell component, wherein the second shell component surrounds the nanoparticle layer;
wherein the precursor comprises at least one compound of Formula (I) or Formula (II), or mixture thereof,
wherein Formula (I) is (M^{v}O_{z}Yₙ)_{w},
wherein Formula (II) is (M^{v}O_{z}YₙR¹ₚ)_{w},
wherein for Formula (I), Formula (II), or the mixture thereof:
each M is independently selected from the group consisting of silicon, titanium, and aluminum,
v is the valence number of M and is 3 or 4,
z is from 0.5 to 1.6,
each Y is independently selected from —OH, —OR², halogen, 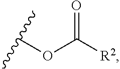
NH₂, —NHR², —N(R²)₂, and 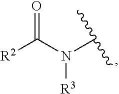
wherein R² is a C₁ to C₂₀ alkyl, C₁ to C₂₀ alkylene, C₆ to C₂₂ aryl, or a 5-12 membered heteroaryl, wherein the heteroaryl comprises from 1 to 3 ring heteroatoms selected from O, N, and S,
wherein R³ is a H, C₁ to C₂₀ alkyl, C₁ to C₂₀ alkylene, C₆ to C₂₂ aryl, or a 5-12 membered heteroaryl, wherein the heteroaryl comprises from 1 to 3 ring heteroatoms selected from O, N, and S,
w is from 2 to 2000;
wherein for Formula (I),
n is from 0.7 to (v-1); and
wherein for Formula (II),
n is from 0 to (v-1);
each R¹ is independently selected from the group consisting of: a C₁ to C₃₀ alkyl; a C₁ to C₃₀ alkylene; a C₁ to C₃₀ alkyl substituted with a member selected from the group consisting of a halogen, —OCF₃, —NO₂, —CN, —NC, —OH, —OCN, —NCO, alkoxy, epoxy, amino, mercapto, acryloyl, —CO₂H, —C(O)-alkyl, —C(O)O-aryl, and —C(O)O-heteroaryl; and a C₁ to C₃₀ alkylene substituted with a member selected from the group consisting of a halogen, —OCF₃, —NO₂, —CN, —NC, —OH, —OCN, —NCO, alkoxy, epoxy, amino, mercapto, acryloyl, —C(O)OH, —C(O)O-alkyl, —C(O)O-aryl, and —C(O)O-heteroaryl; and
p is a number that is greater than zero and is up to pmax,
wherein pmax=60/[9*Mw(R¹)+8],
wherein Mw(R¹) is the molecular weight of the R¹ group.

2. The hair care composition according to claim 1, wherein the precursor comprises at least one compound according to Formula (I).

3. The hair care composition according to claim 1, wherein the precursor comprises at least one compound according to Formula (II).

4. The hair care composition according to claim 1, wherein the one or more capsules are characterized by one or more of the following:
(a) a mean volume weighted capsule diameter of from about 10 μm to about 200 μm, preferably about 10 μm to about 190 μm;
(b) a mean shell thickness of from about 170 nm to about 1000 nm;
(c) a volumetric core/shell ratio of from about 50:50 to 99:1, preferably 60:40 to 99:1, more preferably 70:30 to 98:2, even more preferably 80:20 to 96:4;
(d) the first shell component comprises no more than about 5 wt %, preferably no more than about 2 wt %, more preferably about 0 wt %, of organic content, by weight of the first shell component; or
(e) a mixture thereof.

5. The hair care composition according to claim 1, wherein the compounds of Formula (I), Formula (II), or both are characterized by one or more of the following:
(a) a Polystyrene equivalent Weight Average Molecular Weight (Mw) of from about 700 Da to about 30,000 Da;
(b) a degree of branching of 0.2 to about 0.6;
(c) a molecular weight polydispersity index of about 1 to about 20; or
(d) a mixture thereof.

6. The hair care composition according to claim 1, wherein for Formula (I), Formula (II), or both, M is silicon.

7. The hair care composition according to claim 1, wherein for Formula (I), Formula (II), or both, Y is OR, wherein R is selected from a methyl group, an ethyl group, a propyl group, or a butyl group, preferably an ethyl group.

8. The hair care composition according to claim 1, wherein the second shell component comprises a material selected from the group consisting of calcium carbonate, silica, and a combination thereof.

9. The hair care composition according to claim 1, wherein the inorganic nanoparticles of the first shell component comprise at least one of metal nanoparticles, mineral nanoparticles, metal-oxide nanoparticles or semi-metal oxide nanoparticles.

10. The hair care composition according to claim 9, wherein the inorganic nanoparticles comprise one or more materials selected from the group consisting of SiO₂, TiO₂, Al₂O₃, Fe₂O₃, Fe₃O₄, CaCO₃, clay, silver, gold, or copper.

11. The hair care composition according to claim 10, wherein the inorganic nanoparticles comprise at least one of SiO₂, CaCO₃, Al₂O₃ or clay.

12. The hair care composition according to claim 1, wherein the inorganic second shell component comprises at least one of SiO₂, TiO₂, Al₂O₃, CaCO₃, Ca₂SiO₄, Fe₂O₃, Fe₃O₄, iron, silver, nickel, gold, copper, or clay.

13. The hair care composition according to claim 12, wherein the inorganic second shell component comprises at least one of SiO₂ or CaCO₃.

14. The hair care composition according to claim 13, wherein the inorganic second shell component comprises SiO₂.

15. The hair care composition according to claim 1, wherein the hair care composition comprises from about 5% to about 99.5%, by weight of the composition, of water.

16. The hair care composition according to claim 1, wherein the hair care composition is characterized by a viscosity of from 1 to 1500 centipoises (1-1500 mPa*s), at 20 s⁻¹ and 21° C.

17. The hair care composition according to claim 1, wherein the one or more capsules is present at a level of about 0.1% to about 10%, by weight of the hair care composition.

18. The hair care composition according to claim 1, wherein the hair care composition further comprises a structurant.

19. A personal care composition compromising
a surfactant;
skin conditioning agent, and
a population of capsules;
a capsule comprising a core and a shell surrounding the core;
wherein the core comprises perfume raw materials;
wherein the shell comprises—
a substantially inorganic first shell component comprising a condensed layer and a nanoparticle layer;
wherein the condensed layer comprises a condensation product of a precursor;
wherein the nanoparticle layer comprises inorganic nanoparticles; and
wherein the condensed layer is disposed between the core and the nanoparticle layer;
an inorganic second shell component surrounding the first shell component,
wherein the second shell component surrounds the nanoparticle layer;
wherein the precursor comprises at least one compound of Formula (I) or Formula (II), or mixture thereof,
wherein Formula (I) is (M^{v}O_{z}Yₙ)_{w},
wherein Formula (II) is (M^{v}O_{z}YₙR¹ₚ)_{w},
wherein for Formula (I), Formula (II), or the mixture thereof:
each M is independently selected from the group consisting of silicon, titanium, and aluminum,
v is the valence number of M and is 3 or 4,
z is from 0.5 to 1.6,
each Y is independently selected from —OH, —OR², halogen, 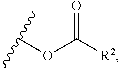
NH₂, —NHR², —N(R²)₂, and 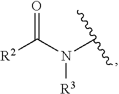
wherein R² is a C₁ to C₂₀ alkyl, C₁ to C₂₀ alkylene, C₆ to C₂₂ aryl, or a 5-12 membered heteroaryl, wherein the heteroaryl comprises from 1 to 3 ring heteroatoms selected from O, N, and S, wherein R³ is a H, C₁ to C₂₀ alkyl, C₁ to C₂₀ alkylene, C₆ to C₂₂ aryl, or a 5-12 membered heteroaryl, wherein the heteroaryl comprises from 1 to 3 ring heteroatoms selected from O, N, and S,
w is from 2 to 2000;
wherein for Formula (I),
n is from 0.7 to (v-1); and
wherein for Formula (II),
n is from 0 to (v-1);
each R¹ is independently selected from the group consisting of: a C₁ to C₃₀ alkyl; a C₁ to C₃₀ alkylene; a C₁ to C₃₀ alkyl substituted with a member selected from the group consisting of a halogen, —OCF₃, —NO₂, —CN, —NC, —OH, —OCN, —NCO, alkoxy, epoxy, amino, mercapto, acryloyl, —CO₂H, —C(O)-alkyl, —C(O)O-aryl, and —C(O)O-heteroaryl; and a C₁ to C₃₀ alkylene substituted with a member selected from the group consisting of a halogen, —OCF₃, —NO₂, —CN, —NC, —OH, —OCN, —NCO, alkoxy, epoxy, amino, mercapto, acryloyl, —C(O)OH, —C(O)O-alkyl, —C(O)O-aryl, and —C(O)O-heteroaryl; and
p is a number that is greater than zero and is up to pmax,
wherein pmax=60/[9*Mw(R¹)+8],
wherein Mw(R¹) is the molecular weight of the R¹ group.

20. The personal care composition according to claim 19, wherein the precursor comprises at least one compound according to Formula (I).

21. The personal care composition according to claim 19, wherein the precursor comprises at least one compound according to Formula (II).

22. The personal care composition according to claim 19, wherein the one or more capsules are characterized by one or more of the following:
(a) a mean volume weighted capsule diameter of from about 10 μm to about 200 μm, preferably about 10 μm to about 190 μm;
(b) a mean shell thickness of from about 170 nm to about 1000 nm;
(c) a volumetric core/shell ratio of from about 50:50 to 99:1, preferably 60:40 to 99:1, more preferably 70:30 to 98:2, even more preferably 80:20 to 96:4;
(d) the first shell component comprises no more than about 5 wt %, preferably no more than about 2 wt %, more preferably about 0 wt %, of organic content, by weight of the first shell component; or
(e) a mixture thereof.

23. The personal care composition according to claim 19, wherein the compounds of Formula (I), Formula (II), or both are characterized by one or more of the following:
(a) a Polystyrene equivalent Weight Average Molecular Weight (Mw) of from about 700 Da to about 30,000 Da;
(b) a degree of branching of 0.2 to about 0.6;
(c) a molecular weight polydispersity index of about 1 to about 20; or
(d) a mixture thereof.

24. The personal care composition according to claim 19, wherein for Formula (I), Formula (II), or both, M is silicon.

25. The personal care composition according to claim 19, wherein for Formula (I), Formula (II), or both, Y is OR, wherein R is selected from a methyl group, an ethyl group, a propyl group, or a butyl group, preferably an ethyl group.

26. The personal care composition according to claim 19, wherein the second shell component comprises a material selected from the group consisting of calcium carbonate, silica, and a combination thereof.

27. The personal care composition according to claim 19, wherein the inorganic nanoparticles of the first shell component comprise at least one of metal nanoparticles, mineral nanoparticles, metal-oxide nanoparticles or semi-metal oxide nanoparticles.

28. The personal care composition according to claim 27, wherein the inorganic nanoparticles comprise one or more materials selected from the group consisting of SiO₂, TiO₂, Al₂O₃, Fe₂O₃, Fe₃O₄, CaCO₃, clay, silver, gold, or copper,

29. The personal care composition according to claim 28, wherein the inorganic nanoparticles comprise at least one of SiO₂, CaCO₃, Al₂O₃ or clay.

30. The personal care composition according to claim 19, wherein the inorganic second shell component comprises at least one of SiO₂, TiO₂, Al₂O₃, CaCO₃, Ca₂SiO₄, Fe₂O₃, Fe₃O₄, iron, silver, nickel, gold, copper, or clay.

31. The personal care composition according to claim 30, wherein the inorganic second shell component comprises at least one of SiO₂ or CaCO₃.

32. The personal care composition according to claim 31, wherein the inorganic second shell component comprises SiO₂.

33. The personal care composition according to claim 19, wherein the personal care composition comprises from about 5% to about 99.5%, by weight of the composition, of water.

34. The personal care composition according to claim 19, wherein the personal care composition is characterized by a viscosity of from 1 to 1500 centipoises (1-1500 mPa*s), at 20 s⁻¹ and 21° C.

35. The personal care composition according to claim 19, wherein the population of encapsulates is present at a level of about 0.1% to about 10%, by weight of the personal care composition.

36. The personal care composition according to claim 19, wherein the personal care composition further comprises a structurant.

37. The personal care composition according to claim 19, where in the personal care composition comprises more than one phase.

38. The personal care composition according to claim 37, wherein the personal care composition comprises a cleansing phase and a benefit phase.

39. The personal care composition according to claim 38, wherein the cleansing phase comprises at least one of surfactant, cationic polymers, associative polymers, emulsifiers, electrolytes, or mixtures thereof.

40. The personal care composition according to claim 38, wherein the benefit phase comprises a benefit agent and at least one additional ingredient.
